SEQUENCE LISTING (3 sequences)

SEQ ID NO: 1 - DNA, 826 bp, Human
gcgttgttgccactccgccaccatgttcgaggcgcgcctggtccagggctccatcctcaagaaggtgttggaggcactcaaggacctcatcaacgaggcctgctgggatattagctccagcggtgtaaacctgcagagcatggactcgtcccacgtctctttggtgcagctcaccctgcggtctgagggcttcgacacctaccgctgcgaccgcaacctggccatgggcgtgaacctcaccagtatgtccaaaatactaaaatgcgccggcaatgaagatatcattacactaagggccgaagataacgcggataccttggcgctagtatttgaagcaccaaaccaggagaaagtttcagactatgaaatgaagttgatggatttagatgttgaacaacttggaattccagaacaggagtacagctgtgtagtaaagatgccttctggtgaatttgcacgtatatgccgagatctcagccatattggagatgctgttgtaatttcctgtgcaaaagacggagtgaaattttctgcaagtggagaacttggaaatggaaacattaaattgtcacagacaagtaatgtcgataaagaggaggaagctgttaccatagagatgaatgaaccagttcaactaacttttgcactgaggtacctgaacttctttacaaaagccactccactctcttcaacggtgacactcagtatgtctgcagatgtaccccttgttgtagagtataaaattgcggatatgggacacttaaaatactacttggctcccaagatcgaggatgaagaaggatcttaggcattcttaaaattcaag

SEQ ID NO: 2 - DNA, 804 bp, Human
atgttcgaggcgcgcctggtccagggctccatcctcaagaaggtgttggaggcactcaaggacctcatcaacgaggcctgctgggatattagctccagcggtgtaaacctgcagagcatggactcgtcccacgtctctttggtgcagctcaccctgcggtctgagggcttcgacacctaccgctgcgaccgcaacctggccatgggcgtgaacctcaccagtatgtccaaaatactaaaatgcgccggcaatgaagatatcattacactaagggccgaagataacgcggataccttggcgctagtatttgaagcaccaaaccaggagaaagtttcagactatgaaatgaagttgatggatttagatgttgaacaacttggaattccagaacaggagtacagctgtgtagtaaagatgccttctggtgaatttgcacgtatatgccgagatctcagccatattggagatgctgttgtaatttcctgtgcaaaagacggagtgaaattttctgcaagtggagaacttggaaatggaaacattaaattgtcacagacaagtaatgtcgataaagaggaggaagctgttaccatagagatgaatgaaccagttcaactaacttttgcactgaggtacctgaacttctttacaaaagccactccactctcttcaacggtgacactcagtatgtctgcagatgtaccccttgttgtagagtataaaattgcggatatgggacacttaaaatactacttggctcccaagatcgaggatgaagaaggatcttaggcattcttaaaattcaag

SEQ ID NO: 3 - DNA, 800 bp, Human
atgttcgaggcgcgcctggtccagggctccatcctcaagaaggtgttggaggcactcaaggacctcatcaacgaggcctgctgggatattagctccagcggtgtaaacctgcagagcatggactcgtcccacgtctctttggtgcagctcaccctgcggtctgagggcttcgacacctaccgctgcgaccgcaacctggccatgggcgtgaacctcaccagtatgtccaaaatactaaaatgcgccggcaatgaagatatcattacactaagggccgaagataacgcggataccttggcgctagtatttgaagcaccaaaccaggagaaagtttcagactatgaaatgaagttgatggatttagatgttgaacaacttggaattccagaacaggagtacagctgtgtagtaaagatgccttctggtgaatttgcacgtatatgccgagatctcagccatattggagatgctgttgtaatttcctgtgcaaaagacggagtgaaattttctgcaagtggagaacttggaaatggaaacattaaattgtcacagacaagtaatgtcgataaagaggaggaagctgttaccatagagatgaatgaaccagttcaactaacttttgcactgaggtacctgaacttctttacaaaagccactccactctcttcaacggtgacactcagtatgtctgcagatgtaccccttgttgtagagtataaaattgcggatatgggacacttaaaatactacttggctcccaagatcgaggatgaagaaggatcttaggcattcttaaaatt

(19) United States
(12) Patent Application Publication
(10) Pub. No.: US 2006/0073477 A1
(43) Pub. Date: Apr. 6, 2006

(54) Altered DNA synthesome components as biomarkers for malignancy

(76) Inventors: Linda H. Malkas, Abingdon, MD (US); Robert J. Hickey, Abingdon, MD (US); Pamela E. Bechtel, Tempe, AZ (US)

Correspondence Address: WHITEFORD, TAYLOR & PRESTON, LLP;ATTN: GREGORY M STONE, SEVEN SAINT PAUL STREET, BALTIMORE, MD, 21202-1626, US

(21) Appl. No.: 10/238,871

(22) Filed: Sep. 11, 2002

Related U.S. Application Data
(62) Division of application No. 09/508,460, filed on May 22, 2000, now abandoned.
(60) Provisional application No. 60/085,200, filed on May 12, 1998.
(60) Provisional application No. 60/060,249, filed on Sep. 29, 1997.

(51) Int. Cl. C12Q 1/68; C07K 16/40

(52) U.S. Cl. 435/6; 530/388.26

(57) ABSTRACT

Antibodies which specifically bind to components of the DNA synthesome which are altered in malignant cells are disclosed. These antibodies can be used, inter alia, to diagnose, prognoses, and treat malignancy and in assays to screen cells, tissues, and body fluids for the presence of a malignant phenotype. These antibodies can be further used to identify test compounds having the ability to suppress the malignant phenotype in a cell by assaying for the ability to inhibit or block the function of an altered component of the DNA synthesome associated with the malignant phenotype. Further, disclosed herein are methods and kit for minimally invasively detecting the presence of neoplasms and malignant conditions using easily obtainable body fluids, such as blood, plasma, lymph, pleural fluid, spinal fluid, saliva, sputum, urine, and semen, for example, to both detect the presence of cancer as well as assess the stage of the disease and the prognosis of the patient. By detecting the presence of an altered form of a component of the DNA synthesome in body fluid, one can diagnose and prognose malignancy. The disclosed method and kit therefor can be used as a diagnostic biomarker for malignancy as well as a means of monitoring the progress and effectiveness of therapeutics.

### CROSS-REFERENCE TO RELATED APPLICATIONS

This application claims priority to U.S. provisional patent application Ser. No. 60/060,249 filed Sep. 29, 1997 and Ser. No. 60/085,200 filed May 12, 1998. Additionally, this application is a continuation-in-part of co-pending U.S. patent application Ser. No. 09/045,624 filed Mar. 20, 1998, entitled “Assay for Measuring the Activity and Fidelity of DNA Replication and Kit Therefor” and U.S. Ser. No. 09/058,760 filed Apr. 11, 1998, entitled “Isolation and Purification of the DNA Synthesome” The contents of these applications are hereby incorporated by reference in their entirety.

### FIELD OF THE INVENTION

The invention relates to the area of cell proliferation, DNA replication, DNA Repair and molecular abnormalities associated with malignant cells and tissues. More particularly, the invention relates to the detection and treatment of malignant cells.

The development of the present invention was supported by the University of Maryland, Baltimore, Md. The invention described herein was supported by funding from the National Institutes of Health (NIH CA 65754 and CA 73060). The Government has certain rights.

### BACKGROUND OF THE INVENTION

One of the critical regulatory points controlling mammalian cell proliferation occurs at the level of DNA replication. Inappropriate levels or timing of DNA synthetic activity result in abnormal cell proliferation and can lead to a variety of undesirable conditions. These conditions range from benign proliferative disorders to lethal neoplasms. Effective treatment for malignancy often depends on the ability to detect reliably the presence of malignant cells at early stages of a disease so that an effective treatment can begin at a stage when the disease is most susceptible to such treatment. Thus, there is a need in the art for reliable techniques for the early detection and treatment of malignant cells.

One major advance in the field of cancer treatment has been the development of improved methods and tests for the early identification of tumors. In many cases these methods lead to the identification of possible malignancies long before they are palpable. Such early detection methodologies include improved diagnostic X-ray techniques, CAT scans, and immunologically based tests using high-affinity antibodies capable of reliably detecting cancer cell specific antigens. Examples of just three such antibody-based tests include those for bladder cancer, prostate cancer, and specific GI tract cancers such as colon cancer. Positive antibody reactivity derived from such tests are usually indicative of the need for further examination of the patient with potential biopsy of suspected tumor sites and subsequent histological examination.

The determination of whether a biopsy sample is benign versus malignant is generally made following histological examination. This method examines the cellular features and anatomical architecture of the biopsy material. The parameters examined at this stage include: % mitotic figures, the apparent differentiation state of the cells, cell ploidy, the level of PCNA expression, % S phase cells, the presence of blood vessels within the specimen, and the regularity of the anatomical boundaries. Tumors with one or more parameters characteristic of malignant tumors are noted and a recommendation is made for tissue resection. This approach often results in the removal of benign tumors as well as malignant tumors. (For example, between 85% and 90% of potential breast tumors first imaged by mammography are found to be benign following surgical resection.) Once tumors are removed they are subjected to a series of specific tests to determine the stage of the tumor and gauge specific cellular features such as expression of specific receptors, mutations in specific genes, microsatellite instability, chromosome translocations, etc. This information combined with the biopsy and diagnostic information available to the physician usually determines the course of treatment and can he used to determine the prognosis of the patient. While these advances in imaging and identification techniques of potential malignancies are responsible for having reduced the overall number of cancer-related deaths, they often lead to unnecessary surgeries and lengthy hospital stays. There is therefore a need for a rapid, minimally invasive technique that can reliably detect potentially malignant cells. There is also a need to be able to reliably detect a potentially malignant cell that has not progressed to the histological stage recognized as malignant, but which can progress to a malignant state.

### SUMMARY OF THE INVENTION

It is an object of the invention to use altered components of the DNA synthesome found in malignant cells, tissues and body fluids as a biomarker for the presence of malignancy. Further to this aim, it is an object of the invention to provide a minimally invasive method and kit therefor to aid in diagnosing or prognosing neoplasm, specifically malignant neoplasm. An alteration in the DNA synthesome of a cell, tissue or body fluid sample obtained from a patient suspected of having a malignant condition is detected. The identification of an altered form of the DNA synthesome or one of its components indicates the presence of a malignant condition in the patient.

It is a further object of the invention to provide a non-invasive method of detecting metastatic malignancy. A body fluid sample of a patient suspected of having metastatic neoplasm is contacted with an antibody which specifically binds to a component of the DNA synthesome which is altered under malignant conditions. Specific binding of the antibody to the cells in the body fluid sample indicates the presence of malignant cells in the sample.

The objects described above may be accomplished using readily available, easily obtainable body fluids, such as blood, plasma, lymph, pleural fluid, spinal fluid, saliva, sputum, urine, and semen, for example.

It is an object of the invention to provide an isolated and purified preparation of antibodies that specifically recognize or bind to components of the DNA synthesome.

It is another object of the invention to provide a method to aid in diagnosing or prognosing malignancy. It is a further object of the invention to provide a kit for diagnosing or prognosing malignancy.

It is yet another object of the invention to provide a method of detecting the presence of malignancy It is a further object of the invention to provide a method of detecting the presence of metastatic malignancy.

It is even another object of the invention to provide a method of screening test compounds for the ability to suppress a malignant phenotype of a cell. It is still another object of the invention to provide a kit for screening test compounds for the ability to suppress a malignant phenotype of a cell.

It is another object of the invention to provide a method of restoring normal function of a DNA synthesome in a malignant cell. It is yet another object of the invention to provide a therapeutic composition for restoring normal function of a DNA synthesome in a malignant cell.

These and other objects of the invention are provided by one or more of the embodiments described below.

One embodiment of the invention provides an isolated and purified preparation of antibodies which specifically bind a component of a DNA synthesome. The component of the DNA synthesome recognized by the antibodies is altered in a malignant cell.

Another embodiment of the invention provides a method to aid in diagnosing or prognosing malignancy. An alteration in a DNA synthesome of a tissue sample whose cells are suspected of being malignant is detected. The identification of an alteration in the DNA synthesome indicates the presence of a malignant cell in the tissue sample.

Another embodiment of the invention provides a method for detecting the presence of malignancy in a patient using imaging techniques. An altered component of the DNA synthesome associated with the malignant phenotype can be labeled so as to be visible by imaging methods well known in the art. The identification or detection of the altered DNA synthesome indicates the presence of a malignant cell in the patient.

Another embodiment of the invention provides a method of detecting the presence of metastatic malignancy. A blood sample of a patient suspected of having a metastatic neoplasm is contacted with an antibody which specifically binds to a component of a DNA synthesome which is altered in a malignant cell. A pattern of specific binding of the antibody to cells in the blood is observed. Specific binding of the antibody to the cells indicates the presence of malignant cells in the blood sample.

Another embodiment of the invention provides a method of screening test compounds for the ability to suppress a malignant phenotype of a cell. A malignant cell is contacted with a test compound. An altered property of a DNA synthesome in the malignant cell is observed. A test compound which restore the altered component of the DNA synthesome in the malignant cell is a potential therapeutic agent for treating malignancy.

Another embodiment of the invention provides a kit for diagnosing or prognosing malignancy. The kit comprises an antibody which specifically binds to a component of a DNA synthesome which is altered in a malignant cell.

Another embodiment of the invention provides a kit for screening test compounds for the ability to suppress a malignant phenotype of a cell. The kit comprises an isolated and purified antibody and a sample of viable malignant cells. The isolated and purified antibody specifically binds to a component of a DNA synthesome which is altered in the malignant cell.

Another embodiment of the invention provides a method of restoring normal function of a DNA synthesome in a malignant cell. The cell is contacted with an antibody which specifically binds to a component of the DNA synthesome which is altered in the malignant cell, or the antibody binding to a cellular component that participates in the regulation of the DNA synthesome activity, thereby altering the activity of the regulator and restoring the normal function of the synthesome. The normal function of the DNA synthesome is restored as a result of the antibody-protein interaction.

Another embodiment of the invention provides a therapeutic composition for restoring normal function of a DNA synthesome in a malignant cell. The therapeutic composition comprises an antibody which specifically binds to a component of the DNA synthesome which is altered in the malignant cell and a pharmacologically suitable excipient.

### BRIEF DESCRIPTION OF THE DRAWINGS

FIG. 1 is a schematic drawing of a DNA synthesome.

FIGS. 2A and 2B depict two-dimensional polyacrylamide gel resolutions of proteins from purified DNA synthesomes isolated from malignant (MCF-7) and nonmalignant (primary breast epithelial cells) breast cell lines, respectively. FIG. 2A shows resolved proteins from MCF-7 synthesomes. FIG. 2B shows resolved proteins from nonmalignant primary breast epithelial cell synthesomes. Components of the synthesome preparations with identical electrophoretic mobilities in each cell type are circled. All other components are altered in the malignant phenotype.

FIG. 3 depicts in vitro levels of DNA replication fidelity of DNA synthesomes isolated from malignant and nonmalignant breast cells.

FIG. 4 depicts in vitro levels of DNA replication activity of DNA synthesomes isolated from malignant and nonmalignant breast cells.

FIGS. 5A-5E are Western blots depicting migration patterns of PCNA isolated from DNA synthesomes from malignant and nonmalignant cells. FIG. 5A shows the migration pattern of PCNA isolated from the DNA synthesome of malignant MCF-7 cells. FIG. 5B shows the migration pattern of PCNA isolated from the DNA synthesome of nonmalignant MCF-10A cells. FIG. 5C shows the migration pattern of PCNA isolated from the DNA synthesome of malignant Hs587T cells. FIG. 5D shows the migration pattern of PCNA isolated from the DNA synthesome of nonmalignant primary breast cells. FIG. 5E shows the migration pattern of PCNA isolated from the DNA synthesome of malignant MDA-MB468 cells.

FIG. 6A-6F are Western blots depicting migration patterns of PCNA isolated from DNA synthesomes from malignant and nonmalignant breast tissues. FIGS. 6A and 6B show the migration pattern of PCNA isolated from human ductal tumors. FIGS. 6C and 6D show the migration pattern of PCNA isolated from human lobular tumor. FIG. 6E shows the migration pattern of PCNA isolated from nonmalignant human breast tissue FIG. 6F shows the migration pattern of PCNA isolated from mouse breast tumor.

FIGS. 7A-7C are Western blots depicting the protein migration pattern of PCNA from A1N4 (FIG. 7A), A1N4myc (FIG. 7B), and A1N4T (FIG. 7C) cells. The parental cell line, A1N4 shown in FIG. 7A, does not contain the cancer specific, acidic form of PCNA. However, the non-malignant cell lines transformed with the c-myc gene and the SV40 T antigen, A1N4myc and A1N4T, do contain the cancer specific form of PCNA.

FIG. 8A-8B are Western blots depicting the protein migration pattern of PCNA from prostate cancer cell lines, LnCAP (FIG. 8A) and PC 10 (FIG. 8B). The malignant prostate cells contain the cancer specific form of PCNA.

FIG. 9A-9C are Western blots depicting the protein migration pattern of PCNA from malignant esophageal-colon cell lines, KGE 90 (FIG. 9A), KYE 350 (FIG. 9B), and SW48 (FIG. 9C). The malignant cells all contain the cancer specific form of PCNA.

FIG. 10A-10C are Western blots depicting the protein migration pattern of PCNA from other cancer cell lines, HeLa—cervical cancer (FIG. 10A), T98—malignant glioma (FIG. 10B) and HL60—promyelogenous leukemia (FIG. 10C). The malignant cells all contain the cancer specific form of PCNA.

FIGS. 11A-11C are Western blots depicting migration patterns of PCNA isolated from DNA synthesomes from estrogen treated MCF-7 cells, control MCF-7 cells, and a benign breast tumor. FIG. 1I A shows the migration pattern of PCNA isolated from estrogen treated MCF-7. FIG. 1I B shows the migration pattern of PCNA isolated from control MCF-7 cells. FIG. 11C show the migration pattern of PCNA isolated from a benign breast tumor.

FIGS. 12A-12C show the nucleotide sequence for the PCNA cDNA clones from MCF-7 and MCF-10A cells. The nucleotide sequence for PCNA cDNA clones from the breast cell lines is aligned with the sequence reported for an acute lymphoblastic leukemia cell. The PCNA nucleotide sequences shown are those of MOLT-4 (12A, SEQ ID NO.: 1): MCF-7 (12B, SEQ ID NO.: 2); and MCF-10A (12C, SEQ ID NO.: 3). Underlined sequences indicate the positions of the ATG start codon and the internal EcoRI restriction endonuclease cleavage site.

FIG. 13 is Western blot illustrating the unique form of PCNA in malignant breast cells (from malignant MCF-7 cells) is not poly-(ADP)-ribosylated.

FIGS. 14A-14E are Western blots depicting the protein migration pattern of PCNA from blood or serum samples taken from cancer patients. All contain the cancer specific, acidic form of PCNA. FIG. 14A shows the serum sample from a patient with intraductal breast cancer. FIG. 14B depicts the blood sample from a patient with acute myelogenous leukemia (AML). FIGS. 14C-14E depict the blood samples from patients with chronic myelogenous leukemia (CML).

FIG. 15 depicts Western blots of the protein migration pattern of PCNA from serum samples taken from control, cancer free patients. The acidic form of PCNA was not detected.

FIG. 16A-16B are Western blots depicting the protein migration pattern of Polymerase α isolated from malignant and nonmalignant human breast cells. FIG. 16A shows the migration pattern of Polymerase α isolated from the DNA synthesome of malignant MCF-7. FIG. 16B shows the migration pattern of Polymerase α isolated from the DNA synthesome of nonmalignant MCF-10A. The malignant cell contain the altered (acidic) form of Polymerase α.

FIG. 17A-17B are Western blots depicting the protein migration pattern of RP-A isolated from malignant and nonmalignant human breast cells. FIG. 17A shows the migration pattern of RP-A isolated from the DNA synthesome of malignant MCF-7. FIG. 17B shows the migration pattern of RP-A isolated from the DNA synthesome of nonmalignant MCF-10A. The malignant cell contain the altered (70 kDa) form of RP-A.

FIG. 18 depicts the results of co-purification assays with the peak of DNA synthesome activity (fraction 5). The results show that both replication and repair proteins are components of the DNA synthesome complex.

FIG. 19 depicts the results of electrophoretic mobility shift assays (EMSAs) with the peak of DNA synthesome activity (fraction 5). FIG. 19A represents incubation with insertion/deletion loop of 2 nucleotides. FIG. 19B represents incubation with insertion/deletion loop of 4 nucleotides. FIG. 19C represents incubation with a G/T mispair. FIG. 19D represents incubation with an A/GO mispair. The top shifted band denotes that the DNA synthesome is bound to the radiolabeled DNA template, thus impeding its mobility through a non-denaturing polyacrylamide gel.

FIG. 20 depicts the results a typical result of the homopolymer competition assay, the assay using labeled heteroduplex template containing a G/T mismatch and unlabeled competitor (identical to the heteroduplex DNA sequence in all matched positions).

### DETAILED DESCRIPTION OF THE PREFERRED EMBODIMENTS

It is a discovery of the present invention that components of the DNA synthesome are altered in malignant cells, and that antibodies which specifically bind to these altered components are useful for the detection and treatment of these malignant cells.

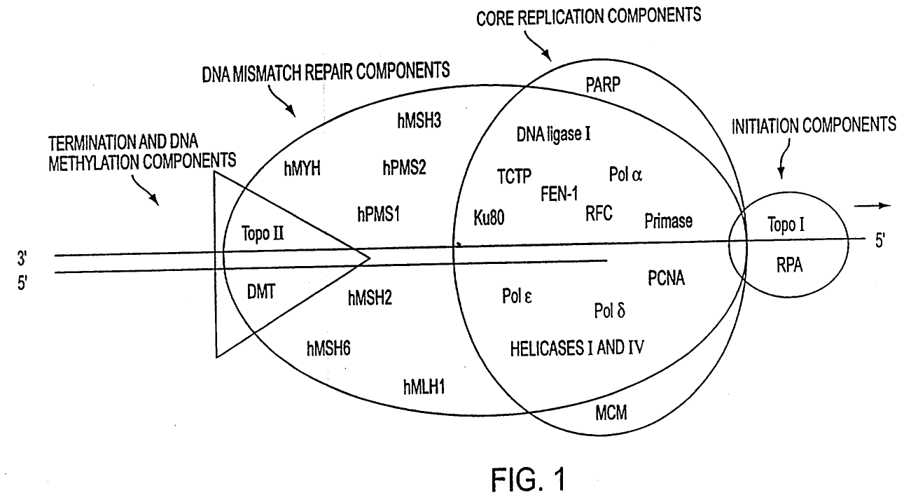

The DNA synthesome is a multiprotein DNA replication complex which is present in mammalian cells (Hickey et al., DNA Replication and Mutagenesis, American Society for Microbiology, pp. 41-54, 1988; Malkas et al., Biochem. 29: 6362, 1990; Applegren et al., J. Cell. Biochem. 59: 91, 1995; Lin et al., Leuk. Res. 21(6): 501-12, 1997; Coll et al, Oncology Research 8 (10/11) 43547, 1996; Hickey and Malkas, Critical Reviews in Eukaryotic Gene Expression 761(2), 125-7, 1997; Tom et al., J. Cell. Biochem. 63, 259-67 1996). The DNA synthesome comprises at least 35 proteins, including DNA polymerase alpha, DNA primase, helicase I, helicase IV, DNA ligase I, topoisomerase I, topoisomerase II, DNA polymerase delta, RPA, poly(ADP-ribose) polymerase, RF-C (Activator-1), polymerase E, and proliferating cell nuclear antigen (PCNA) (see co-pending U.S. patent application Ser. No. 09/058,760 as well as Coll, et al., Oncology Research, 9:629-637, 1997). Further to previous studies, the inventors have discovered that the following additional components are demonstrated to be part of the DNA synthesome: DNA methyltransferase, hMSH2 (homologue of bacterial Mut S), hMLH1 (homologue of bacterial Mut L), hMSH6 (GTBP—homologue of bacterial Mut S), hPMS1 (homologue of yeast post mitotic segregation protein 1), hPMS2 (homologue of yeast post mitotic segregation protein 2), MYH (homologue of bacterial Mut Y), Ku80, MCM (minichromosomal maintenance protein), TCTP (translationally controlled tumor protein) and FEN-1 (see FIG. 1). Furthermore, the following DNA replication, repair, cell cycle and nucleotide metabolism proteins are demonstrated to clearly not be associated with the DNA synthesome: DNA polymerase B, P53, BRCA1, BRCA2, RB, TFII(H), XPA, RNA polymerase II, Annexin I, Annexin II, Dihydrofolate reductase (DHFR), Thymidine Kinase, Thymidilate Synthetase, Thymidilate Kinase, and Nucleotide Diphosphokinase.

The mammalian DNA synthesome is a highly organized structure. The integrity of the multiprotein complex is maintained after its treatment with detergents, salt, RNase, DNase, chromatography on DE52-cellulose or Q-Sepharose, sedimentation in glycerol and sucrose density gradients, and electrophoresis through native polyacrylamide gels (see co-pending U.S. patent application Ser. No. 09/058,760 and Coll et al., Oncol. Res. 8:435-447, 1996; Wu et al., 1994) Further to previous studies, the inventors have discovered that the DNA synthesome complex contains specific repair proteins as listed above. This complex of proteins is fully competent to replicate DNA in vitro (Applegren et al., 1995; Lin et al., 1996; Tom et al., 1996). In vitro replication requires Mg⁺⁺, ribonucleotide and deoxyribonucleotide triphosphates, SV40 large T antigen, a double stranded DNA template containing an SV40 origin of replication, and a renewable source of ATP.

Alternatively, DNA synthesomes can be purified from any mammalian cell type, such as breast epithelial cells or HeLa cells, using the method described in Malkas et al., 1990, Coll et al, 1996, and Applegren et al., 1995. The purified DNA synthesomes can be used as a starting material for the preparation of purified altered components. The DNA synthesome preparation is about 5200-fold purified, thereby resulting in an increase in specific activity.

A purified preparation of the abnormal component is at least 80% pure. Preferably, the preparations are about 90% to about 99% pure, more preferably 95% to 99% pure. Purity of the preparations can be assessed by any means known in the art, such as SDS-polyacrylamide gel electrophoresis. The purified abnormal component can then be used as an immunogen, to prepare polyclonal or monoclonal antibodies using standard procedures known in the art.

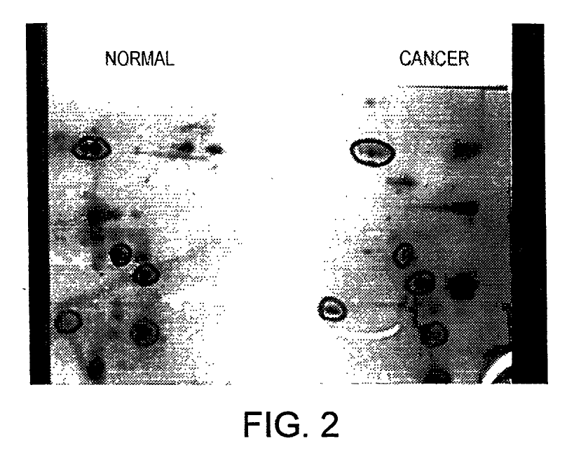

It was discovered that transformation of a nonmalignant cell to a malignant state is accompanied by a significant alteration in the abundance and/or mobility of at least half of the protein components of the DNA synthesome (see FIG. 2). Altered components of the DNA synthesome are protein components of the synthesome whose amino acid or gene sequences, post-translational modifications, or altered expression levels, for example, are altered in malignant cells compared with the corresponding components in nonmalignant cells.

Nonmalignant cells are cells found in mammalian tissues or cell cultures which exhibit typical morphological and temporal patterns and levels of DNA synthesis or cell division. Malignant cells include cells whose levels of DNA synthesis and cell division are higher or occur at atypical times compared with cells in the corresponding normal tissue or cell line

The malignant phenotype develops as the result of a multistep process, requiring the accumulation of multiple genetic mutations. One mechanism through which genetic alterations may occur involves the cellular DNA replication process becoming error prone (Sekowski et al, 1998). An increase in the error frequency associated with the DNA synthetic machinery responsible for elongating the DNA could lead to an accumulation of mutations in the malignant cell through the development of error prone DNA replication process. Since DNA replication is orchestrated by the DNA synthesome complex, alterations of any of the components of the synthesome can correlate to a decrease in replication fidelity.

Amino acid alterations which can be present in an altered component of a DNA synthesome include conservative or non-conservative amino acid substitutions, deletions, or additions. Post-translational modifications which can be observed include, but are not limited to, the presence or absence of ribosylation, glycosylation, sulfation, myristilation, phosphorylation, or the alteration of intramolecular bonds. Expression levels of such altered components of the synthesome can vary from undetectable in non-malignant cells to thousands of copies per malignant cell. Likewise, gene alterations can result in protein truncation.

Components of the DNA synthesome which are altered in malignant cells include, but are not limited to, proliferating cell nuclear antigen (PCNA), DNA polymerase alpha (Pol A), and replication protein A (RP-A). Many of these alterations can be detected in a silver-stained two-dimensional polyacrylamide gel which has been used to separate protein components of the DNA synthesome purified from malignant tissues or cell lines. The alterations include differences in the abundance or position of synthesome components compared with the corresponding components isolated from nonmalignant tissues or cell lines (FIGS. 2A and 2B).

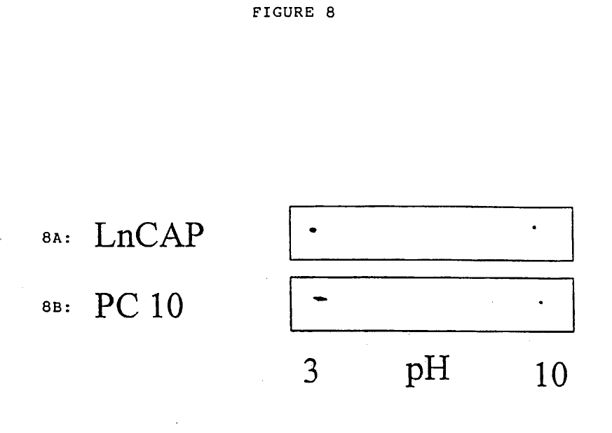

One such protein which is altered in a malignant cell is proliferating cell nuclear antigen (PCNA) (Bechtel et al., Cancer Research, 58:3264-3269, 1998). PCNA is a 36 kD protein which is an accessory factor required by DNA polymerase delta to mediate highly efficient and processive DNA replication activity (Hickey and Malkas, 1996). The DNA synthesome purified from a malignant cell contains two forms of PCNA. The two forms have the same molecular weight, as measured on a Western blot of a two-dimensional polyacrylamide gel stained with a commercially available antibody which specifically binds to PCNA (PC 10, Oncogene Science). However, the two species of PCNA differ significantly in their overall charge (see FIG. 8 and Example 4). Thus, an acidic (malignant) and a basic (nonmalignant) species of PCNA can be readily distinguished on a two-dimensional polyacrylamide gel.

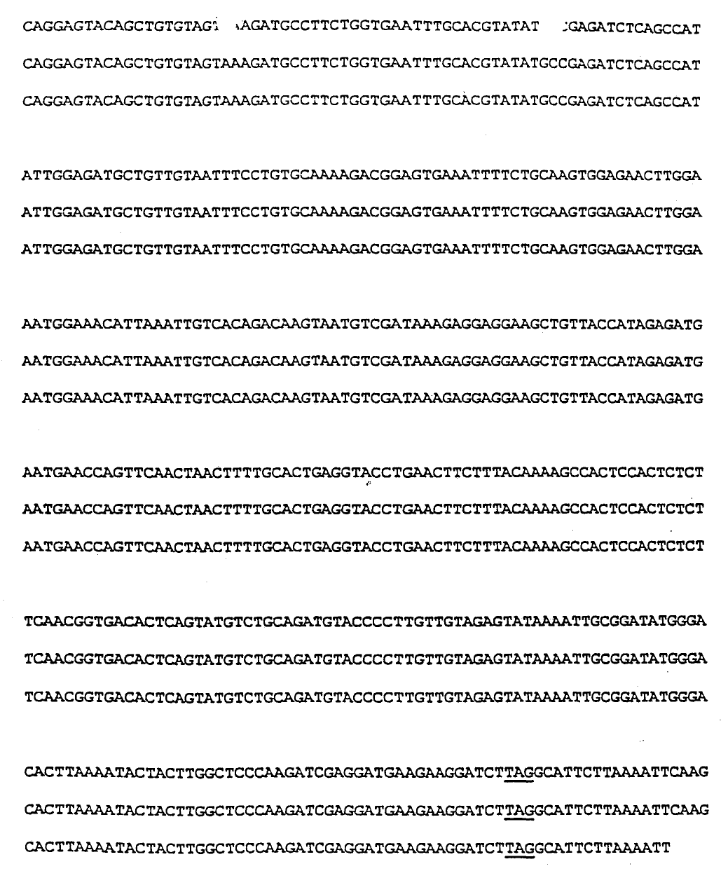

The altered mobility of the acidic PCNA species is due to the loss of a post-translational modification comprising a lack of poly(ADP) ribosylation (see FIG. 13 and Example 4, below). Approximately half of the polypeptides composing the synthesome are post-translationally modified by poly(ADP) ribosylation (Simbulan et al., Bioch. 35(36), 1 1622-33, 1996). While not wishing to be bound by any particular theory, it is hypothesized that poly(ADP) ribosylation of some of the synthesome's components may modulate the synthesome's DNA synthetic activity.

Acidic PCNA is expressed in malignant cell lines, such as HeLa (human cervical carcinoma), Hs578T (breast carcinoma), HL-60 (human promyelogenous leukemia), FM3A (mouse mammary carcinoma), PC 10 (prostate carcinoma), LnCAP (prostate carcinoma), LN99 (prostate carcinoma) MD-MB468 (human breast carcinoma), MCF-7 (breast carcinoma), KGE 90 (esophageal-colon carcinoma), KYE 350 (esophageal-colon carcinoma), SW 48 (esophageal-colon carcinoma) and T98 (malignant glioma). Acidic PCNA is also expressed in malignant cells obtained from human breast tumors, prostate tumors, brain tumors, human gastrointestinal or esophageal-colon tumors, murine breast tumors and in human chronic myelogenous leukemia. Acidic PCNA is not detected in nonmalignant cell lines, such as the breast cell lines Hs578Bst and MCF-10A, or in samples of nonmalignant serum or tissue, such as breast.

Commercially available antibodies do not distinguish between the acidic and basic forms of PCNA. In fact, all the commercially available PCNA antibodies recognize the same epitope. Thus, commercially available anti-PCNA antibodies cannot be used to specifically detect only the malignant form of PCNA. It was discovered, however, that antibodies which specifically bind to altered forms of DNA synthesome components are useful for the detection of malignant cells. The antibodies can be used to detect altered components of the DNA synthesome in tissues or cell lines, as therapeutics, and in assays for screening test compounds for the ability to affect cell proliferation or suppress a malignant phenotype.

The antibodies can be prepared using a variety of methodologies. For example, a purified altered component of a DNA synthesome can be used as an immunogen, to obtain a preparation of antibodies which specifically bind to the altered component. Any method or combination of methods known in the art can be used to purify the desired altered synthesome component including, but not limited to, size exclusion chromatography, ammonium sulfate fractionation, ion exchange chromatography, affinity chromatography, crystallization, electrofocusing, and preparative gel electrophoresis. A spot containing an abnormal synthesome component which can be detected on a two-dimensional polyacrylamide gel, for example, acidic PCNA, can be excised from such a gel, eluted from the polyacrylamide, and purified, as is known in the art. The skilled artisan can readily select methods which will result in a preparation of each abnormal component which is substantially free from other proteins, carbohydrates, lipids, or subcellular organelles.

In a preferred embodiment, the antibodies are prepared using the phage display method (Winter et al., Ann. Rev. Immunol. 12: 433 (1994). A filamentous phage such as M13 can be engineered to express on its surface a fusion protein consisting of a phage coat protein, such as the product of M13 gene 3, and a fragment of an immunoglobulin (ssV region) variable combining region domain. Phage expressing an antigen combining site recognizing the antigen of interest are selected by one of several methods in which (for example) the antigen is immobilized on a fixed support and the support is incubated with the phage library containing phage which express the V gene combining region recognizing the specific antigen of interest. The recombinant phage specifically binding the antigen is isolated by washing the support with a solution of sufficient ionic strength to disrupt the interaction of the immobilized antigen and the phage. The isolated phage are then used to infect E. coli, and the specific phage are further purified by repeating the isolation process using the immobilized antigen. The process is repeated a third time to isolate an essentially homogeneous population of phage recognizing the specific antigen of interest. The phage DNA is then isolated, the insert encoding the V gene region is excised, recloned into an expression plasmid (pSYN1), which expressed c-myc, the Lac Z alpha gene, and a nucleotide sequence encoding a Histidine hexamer. The c-myc product is recognized by an antibody specifically recognizing c-myc, and a nickel spin column is used to affinity purify the antibody combining region. Large scale isolation of the antigen combining region is performed by hypertonic shock of the bacteria transfected with the engineered pSYN1 plasmid, and subsequent passage of the released proteins over a nickel column.

The antibodies of the invention specifically bind to epitopes present on components of the DNA synthesome which are altered in malignancies. Preferably, the epitopes are not present in other mammalian proteins. An epitope typically comprises from about 5 to about 12 contiguous amino acids. However, more amino acids can contribute to an epitope. For example, if the epitope involves noncontiguous residues, then from about 14 to about 50 or more amino acids can comprise the epitope. The presence or absence of post-translational modifications, such as glycosylation or ribosylation, on the DNA synthesome components can also contribute to an epitope. In addition, monoclonal and polyclonal antibodies can be produced by any method known in the art using the purified antigen as described above.

Antibodies which specifically bind to altered DNA synthesome components provide a detection signal from about 2 to about 20-fold higher than a detection signal provided with other proteins when used in Western blots or other immunochemical assays. Preferably, antibodies which specifically bind altered DNA synthesome components do not detect the corresponding unaltered proteins in immunochemical assays and can immunoprecipitate the altered synthesome components from solution.

The antibodies of the invention can be purified by methods well known in the art. Preferably, monoclonal or polyclonal antibodies are affinity purified, by passing antiserum over a column to which the antigenic component of the DNA synthesome is bound. The bound antibodies can then be eluted from the column, for example using a buffer with a high salt concentration or an altered pH.

Malignant cells which can be detected using the antibodies of the invention include, but are not limited to, malignant cells in tissues such as breast, prostate, blood, brain, pancreas, smooth or striated muscle, liver, spleen, thymus, lung, ovary, skin, heart, connective tissue, kidney, bladder, intestine, stomach, adrenal gland, lymph node, or cervix, or in cell lines, for example, Hs578T, MCF7, MDA-MB468, HeLa, HL60, FM3A, BT-474, MDA-MB-453, T98, LnCAP, LN 99, PC 10, SK-OV-3, MKN-7. KGE 90, KYE 350, or SW 48. Thus, the antibodies can be used to diagnose malignancy. The antibodies can also be used to prognose the development of a malignancy, for example, by correlating the levels of one or more altered components with the progression of a particular malignant disease. Furthermore, the antibodies can be used to prognose the potential survival outcome for a patient who has developed a malignancy.

Diseases which can be diagnosed or prognosed using the antibodies of the invention include, but are not limited to, malignancies such as various forms of glioblastoma, glioma, astrocytoma, meningioma, neuroblastoma, retinoblastoma, melanoma, colon carcinoma, lung carcinoma, adenocarcinoma, cervical carcinoma, ovarian carcinoma, bladder carcinoma, lymphoblastoma, leukemia, osteosarcoma, breast carcinoma, hepatoma, nephroma, adrenal carcinoma, or prostate carcinoma, esophageal carcinoma.

The antibodies can also be used to stage malignant tumors, by comparing levels of one or more abnormal DNA synthesome components in a tumor over time, to follow the progression of a malignant disease, or a patient's response to treatment. The antibodies can also be used to detect malignant cells which have broken free from a tumor and are present in a patient's bloodstream, by using the antibodies to assay a blood sample for the presence of the abnormal components. The patients can be either human or veterinary patients.

Cells can be assayed for the presence of an altered component to which an antibody of the invention specifically binds by any means known in the art. For example, tissue sections or cell cultures can be mounted on glass or plastic slides and contacted with an antibody of the invention according to standard immunocytochemical protocols. The antibody can include a detectable label, such as a radioactive, fluorescent, chemiluminescent, enzymatic, or biotinylated moiety. Alternatively, specific binding between the antibody and the altered component can be detected using a secondary antibody. Many systems for the detection of bound antibodies are known in the art. Alternatively, an enzyme linked immunosorbent assay (ELISA) or radioimmunoassay (RIA) can be used to detect specific binding of the antibodies in solubilized cells. The antibodies of the invention can also be used in Western blots of one- or two-dimensional polyacrylamide gels which have been used to separate proteins from the cells or tissues to be tested. Such methods are familiar and widely practiced in the art.

The concentration of antibody to be used will depend on the particular antibody and its affinity for the abnormal component of the DNA synthesome. Typically, antibody affinities are from about 10⁴ M⁻¹ to about 10⁹ M⁻¹. Concentrations of specifically binding antibodies used in the immunochemical methods discussed above can be, for example, approximately 250 to about 2000 nanograms of antibody per ml. Or up to 50-500 μg per ml. In a preferred embodiment, the antibody recognizes only an acidic form of PCNA. Antibodies which specifically bind to acidic PCNA can be used at concentrations of, for example, from about 0.5 μg per ml to about 500 μg per ml.

The antibodies of the invention are also supplied in a kit. The kit can include additional components, for example, reagents such as blocking antiserum, secondary antibodies, buffers, or labeling reagents for carrying out immunochemical staining, ELISAs, or RIAs with the antibodies. The kit can also include instructions for using the kit as a diagnostic or prognostic aid for malignancies.

The antibodies can be used in assays to screen test compounds for the ability to suppress a malignant phenotype of a cell. The assay comprises contacting a malignant cell with a test compound and observing an altered property of a DNA synthesome in the malignant cell. The antibodies of the invention can be supplied in a kit, together with a viable sample of malignant cells, for use in such screening assays. The malignant cell can be from any cell line which expresses an altered property of a DNA synthesome, including, but not limited to. Hs578T, MCF7, MDA-MB-468 HeLa. FM3A, or LN99. The malignant phenotype to be suppressed includes characteristics such as increased proliferation, increased DNA synthetic activity, decreased DNA replication fidelity, altered levels of protein expression, and altered DNA synthesome components.

The altered property of the DNA synthesome can be any property associated with the synthesome in a malignant cell, such as alterations in expression level, amino acid sequence, post-translational modification, or electrophoretic mobility of protein components or levels of DNA synthetic activity or replication fidelity. DNA synthetic activity or replication fidelity can be measured as described below. Preferably, the altered property of the DNA synthesome is an altered component of a DNA synthesome which can be detected using an antibody which specifically binds to the altered component. Most preferably, the altered component is the acidic form of PCNA.

The test compound can be a pharmacologic compound already known in the art to have an effect on a malignant phenotype or other pharmacological effect, or can be a compound previously unknown to have any pharmacological activity. The test compound can be naturally occurring or designed in the laboratory. The test compound can be isolated from a microorganism, animal, or plant, or can be produced recombinantly or synthesized by chemical methods known in the art. A test compound which decreases the expression of the abnormal synthesome component, decreases levels of DNA synthetic activity of a purified synthesome, or increases levels of replication fidelity of a purified DNA synthesome is a potential therapeutic agent for suppressing a malignant phenotype and for treating malignancy.

The antibodies of the invention can also be used as therapeutic agents, to restore the normal function of a DNA synthesome in a malignant cell. The antibodies can be delivered to a malignant cell in a human or veterinary patient using any methods known in the art. For example, full-length antibodies, antibody fragments, or antibody fusion proteins which bind specifically to synthesome proteins which are altered in malignant cells, can be administered to such patients.

Preferably, the therapeutic composition is administered soon after obtaining a positive result using the diagnostic method of the invention. Both the dose and the means of administration of the therapeutic composition can be determined based on the specific qualities of the composition, the condition, age, and weight of the patient, the progression of the particular disease being treated, and other relevant factors. Administration can be local or systemic, including injection, oral administration, catheterized administration, and topical administration.

Preferably, receptor-mediated targeted delivery of therapeutic compositions containing the antibodies of the invention is used to deliver the antibodies to specific tissues. Many tumors, including breast, lung, and ovarian carcinomas, overexpress antigens specific to malignant cells, such as glycoprotein p185^{HER2}. Antibodies which specifically bind to these antigens can be bound to liposomes which contain an antibody of the invention. When injected into the bloodstream of a patient, the anti-p185^{HER2} antibody directs the liposomes to the target cancer cells, where the liposomes are endocytosed and thus deliver their contents to the malignant cell (see Kirpotin et al., Biochem. 36: 66, 1997).

In a preferred embodiment, a p185^{HER2} antibody targeted delivery system is used to deliver an antibody which specifically binds to an acidic PCNA protein in a breast cancer cell. Liposomes can be loaded with the antibody as is known in the art (see Papahadjopoulos et al., Proc. Natl. Acad. Sci. 88: 11640, 1991; Gabizon, Cancer Res. 52: 891, 1992; Lasic and Martin, Stealth Liposomes, 1995; Lasic and Papahadjopoulos, Science 267: 1275, 1995; and Park et al., Proc. Natl. Acad. Sci. 92: 1327, 1995). Such liposomes contain 0.1-0.15 mg of anti-acidic PCNA antibody per μmol liposome and can be administered to patients in a range of about 5 mg/kg. The therapeutic composition can include a pharmacological excipient, such as etoposide or cytosine arabinoside, or adriamycin.

The DNA synthesome purified from malignant cells have a two- to eight-fold lower DNA replication fidelity than do synthesomes purified from cells which proliferate normally. Thus, this functional property of the purified DNA synthesome can also be used to detect malignant cells. DNA replication fidelity can be assessed as taught, for example, in Sekowski et al., Toxicol. Applied Pharmacol. 145: 268 (1997) and Sekowski et al. 1998.

### EXAMPLES

The following are provided for the purpose of exemplification only and are not intended to limit the invention which has been described in broad terms above.

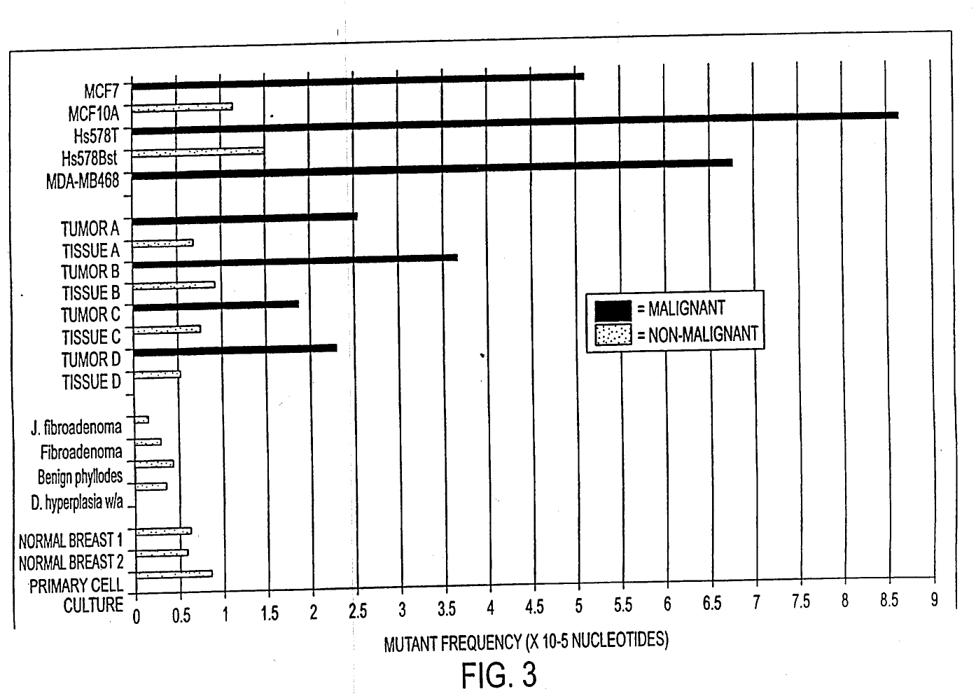

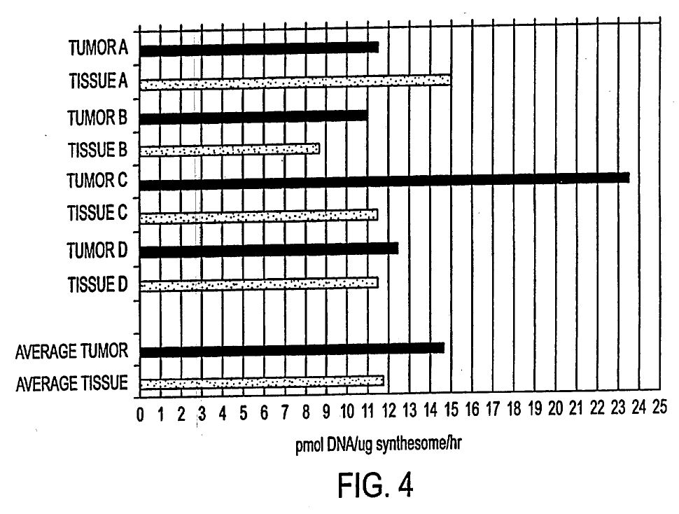

The experiments discussed under Example 1 relate to the replication properties of malignant and nonmalignant DNA synthesomes. The example demonstrates that malignant DNA synthesome mediates an error-prone DNA replication (Examples 1A and 1B). However, malignant and non-malignant DNA synthesome replication activity are relatively similar (Example 1C). Therefore, it is clear that the decrease in replication fidelity is not a result of replication activity. FIGS. 3 and 4 are associated with the findings in Example 1.

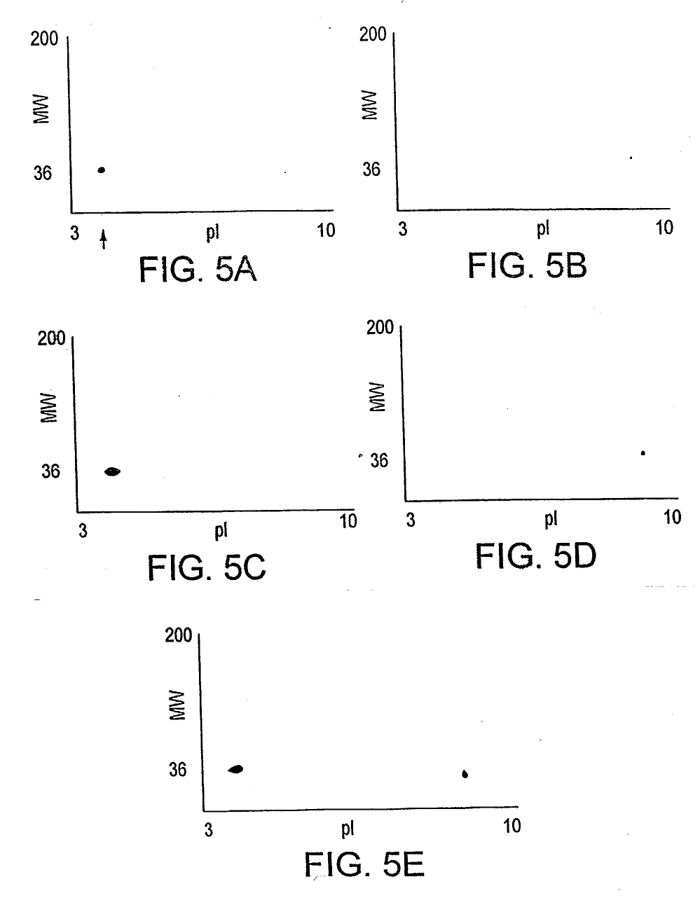

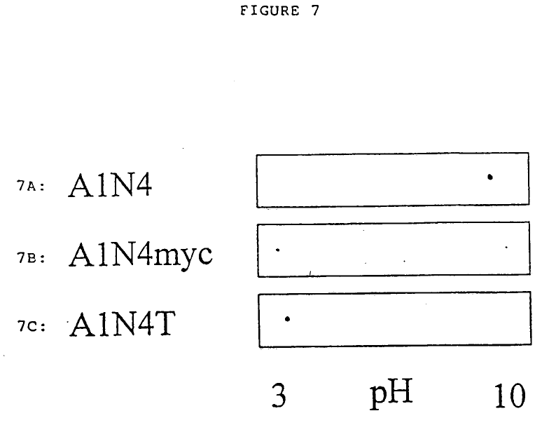

The experiments discussed under Example 2 relate to the discovery of an altered (acidic) form of PCNA in malignant breast cells and tissues. Example 2A focuses on breast cells, examples 2B and 2C on breast tumors and tissues. FIGS. 5 and 7 are associated with the findings in Example 2.

The experiments in Example 3 relate to the discovery of the altered (acidic) form of PCNA in other malignant cells. Example 3A focuses on prostate cancer cells, example 3B focuses on malignant esophageal-colon cells, and example 3C on malignant cells from cervical cancer, brain cancer and leukemia. FIGS. 8 to 10 are associated with the findings in Example 3.

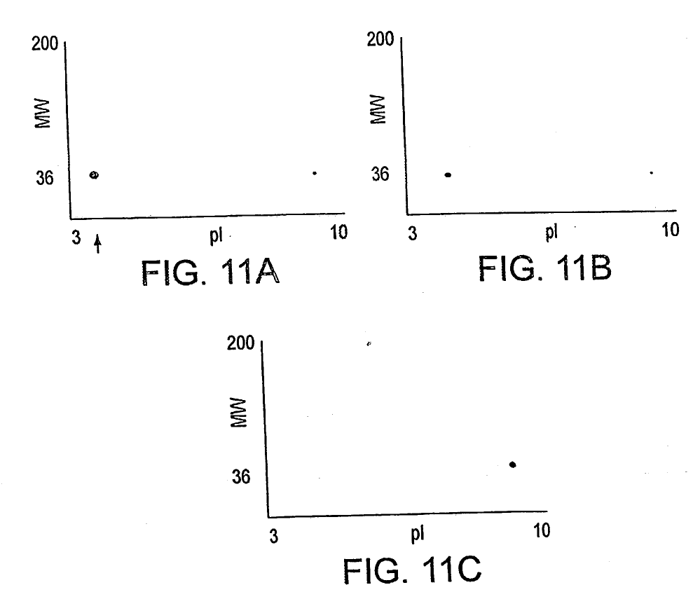

The experiments in Example 4 relate to characterization of the malignant form of PCNA. The results of example 4A indicate that the malignant (acidic) form of PCNA is not poly-ADP-ribosylated. The results of example 4B indicate that the malignant form of PCNA is not a result of cell proliferation. The results of example 4B indicate that the malignant form of PCNA is not a result of genetic mutation. FIGS. 11 to 13 are associated with the findings in Example 4.

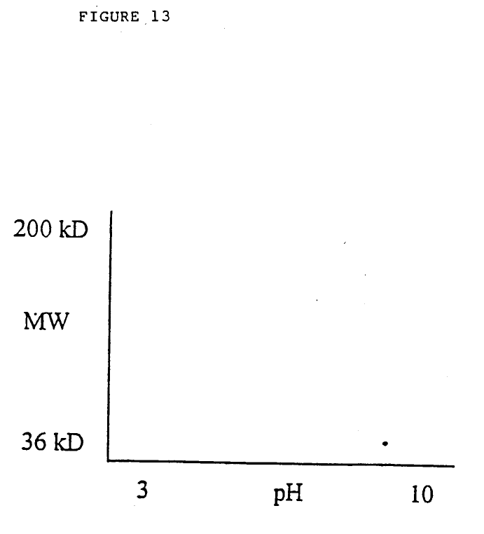

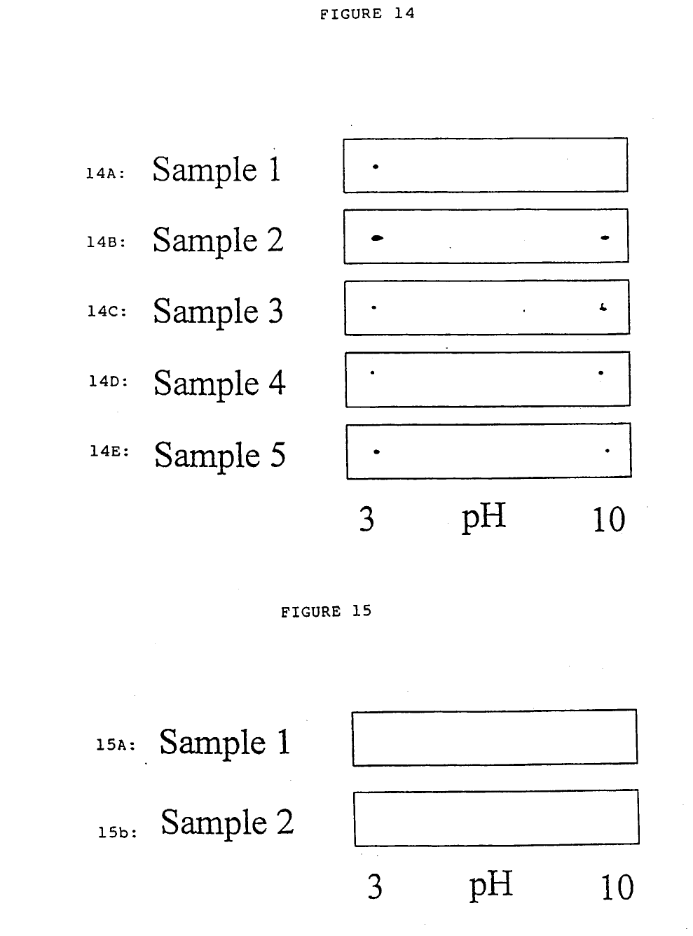

The experiments in Example 5 relate to the discovery of the malignant form of PCNA In body fluids such as blood (Example 5A) and serum (Example 5B). FIGS. 14 and 15 are associated with the findings in Example 5.

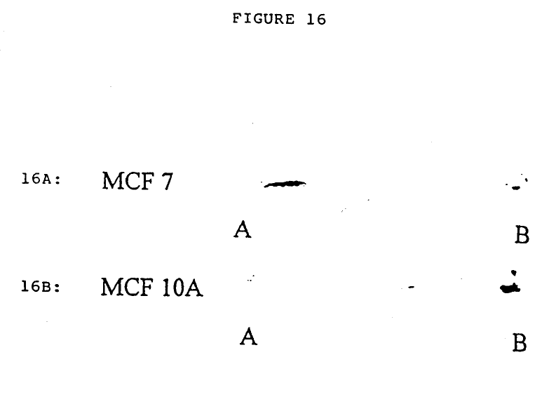

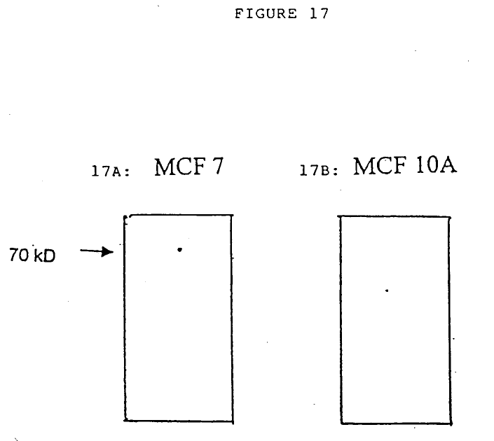

The experiments in Example 6 relate to the discovery of an altered form of other components of the DNA synthesome in malignant cells. Example 6A discusses the altered form of polymerase α found in malignant breast cells yet not in nonmalignant breast cells. Example 6B discussed the altered form of RP-A found in malignant breast cells yet not in nonmalignant breast cells. FIGS. 16 and 17 are associated with the findings in Example 6.

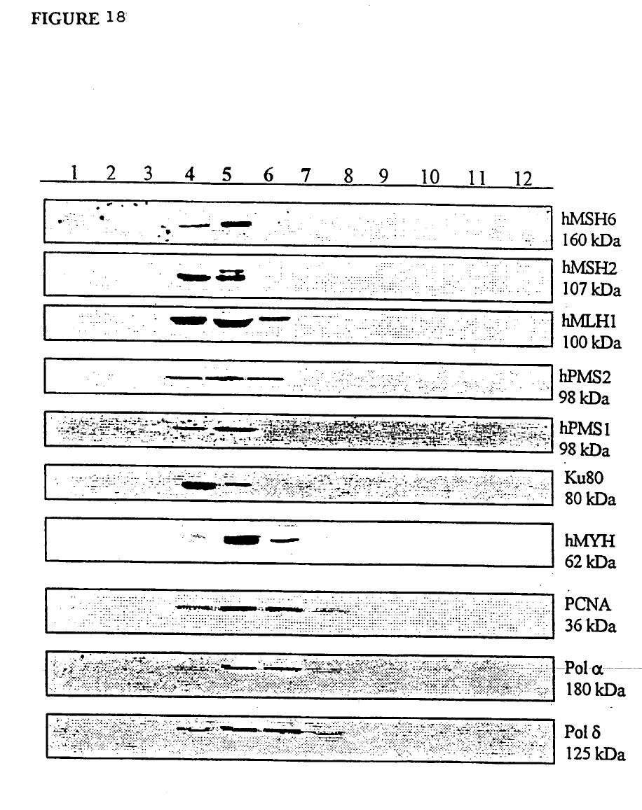

The experiments in Example 7 relate to the determination that the DNA replication components of the DNA synthesome are tightly associated with the DNA repair components. By using co-purification and co-precipitation studies (Example 7A) as well as the homopolymer and heteropolymer competition assays using mismatched DNA templates (Example 7B), the strength of the protein-protein interactions of the components of the synthesome between replication and repair components of the DNA synthesome, are demonstrated. FIGS. 18 to 20 are associated with the findings in Example 7.

### Example 1

### Replication Fidelity and Activity

This example demonstrates that the DNA synthesome derived from malignant breast cell and human breast tumors mediate an error-prone DNA replication. This example further demonstrates that malignant and non-malignant DNA synthesome replication activity are relatively similar, indicating that the decrease in replication fidelity is not a result of replication activity.

### Example 1A

### Human Breast Cells

To assess whether the DNA replication apparatus of malignant breast cells carries out error-prone DNA synthesis, the replication fidelity of the DNA synthesome isolated from malignant and non-malignant human breast cells grown in culture was examined. Using the procedure described in co-pending U.S. patent application Ser. No. 09/058,760, the DNA synthesome from malignant human breast cell lines MDA-MB468, Hs578T, and MCF-7. and the nonmalignant human breast cell lines Hs578Bst and early passage MCF-10A was isolated and purified. The replication fidelity of these preparations was evaluated using the procedure described in co-pending U.S. patent application Ser. No. 09/045,624. The DNA synthesome derived from MCF-7 produced significantly more nucleotide errors in the nascent DNA than did the synthesome of the nonmalignant MCF-10A cells (see Table 1 and FIG. 3). Specifically, the frequency of mutations produced by MCF-7 DNA synthesome was 4.4 fold higher than that created by the DNA synthesome derived from non-malignant MCF-10A cells. Similarly, it was observed that the DNA synthesome derived from malignant Hs578T cells exhibited a 5.7 fold higher DNA replication error frequency than did the synthesome from its genetically matched counterpart, Hs578Bst.

The synthesome from estrogen receptor negative malignant cell line MDA-MB468 also mediated DNA replication using an error-prone mechanism. It was determined that the MDA-MB468 synthesome incorporated errors at a level comparable to that demonstrated by the DNA synthesome from the MCF-7 and Hs578T cell lines. These data indicate that the malignant human breast cell contained an error prone DNA replication apparatus.

### Example 1B

### Human Breast Tissues

To confirm that the results in Example 1A reflected molecular events occurring in human breast tissue, the forward mutagenesis assays described above were performed using the DNA synthesome prepared from surgically resected malignant and nonmalignant human breast tissue were performed. The DNA synthesome was purified by the process described by Stampfer (Stampfer, Tissue Culture Methods, 9:107-115, 1985). To assure that potential differences in replication fidelity were not due to individual genetic variations between patients, the DNA synthesome derived from genetically matched (i.e., the same patient) malignant and nonmalignant tissue from several different breast cancer patients who had not yet received any prior treatment were also examined. The fidelity of replication mediated by the malignant breast tissue DNA synthesome was compared with that carried out by the DNA synthesome derived from genetically matched nonmalignant breast tissue.

The results, also shown in Table 1, show the replication fidelity of the synthesome derived from malignant breast tissue was 2.4 to 4.4 fold lower than that mediated by the genetically matched nonmalignant breast tissue synthesome. Additionally, the level of replication fidelity observed for the synthesome derived from malignant breast tissue was essentially comparable to that of synthesome purified from nonmalignant breast cell cultures. (eg, MCF10A). The fold mutation frequency of the breast tumor tissue synthesome was similar to that observed for the complex derived from the malignant MCF-7 cell cultures. These data indicate that a distinctly error prone DNA replication apparatus is not merely a feature of the cultured breast cancer cells but is also a significant characteristic common to all malignant human breast cells.

### Example 1C

### DNA Replication Activity

To validate that observed increase in the mutation frequency of the purified malignant cell replication apparatus was not merely due to an increase in the level of in vitro DNA synthesis, the DNA replication level mediated by the DNA synthesome derived from genetically matched malignant and nonmalignant breast tissue was examined. The synthesome derived from these different tissues were assayed for their in vitro SV40 DNA replication activity (see co-pending U.S. patent application Ser. No. 09/045,624 and published procedures Coll et al. 1996), the results shown in Table 2 below and FIG. 4. One unit of activity is defined as one picomole of [³²-P]-dCMP incorporated into SV40 origin containing DNA per 2 hours at 35° C. No significant difference in the level of DNA replication activity between malignant and nonmalignant tissues was detected. These data demonstrate that the significant decrease in replication fidelity observed for malignant cell synthesome is not a result of an increased in vitro DNA replication activity exhibited by the replication complex.

TABLE 1
DNA replication fidelity of the malignant and nonmalignant breast synthesome

| | Total no. of | No. of mutant | Mutant frequency | Fold mutation |
|---|---|---|---|---|
| Source of DNA synthesome | colonies scored | colonies | (×10⁻⁵ nucleotides)^{a} | frequency^{b} |
| Malignant | | | | |
| Human breast cells | | | | |
| MCF7 | 6.0 × 10⁴ | 576 | 5.15 | 4.4 |
| Hs578T | 6.0 × 10⁴ | 960 | 8.65 | 5.7 |
| MDA-MB468 | 6.0 × 10⁴ | 762 | 6.81 | N/A^{c} |
| Tumor A^{d} (IDC^{e}) | 3.4 × 10⁴ | 141 | 2.52 | 3.6 |
| [ER −, PR −, Ki-67 24% (high), HER-2/neu 52% | | | | |
| (high), p53 positive] | | | | |
| Tumor B (IDC) | 3.0 × 10⁴ | 209 | 3.72 | 3.8 |
| (ER +, PR +, diploid, 6% S-phase) | | | | |
| Tumor C (ILC) [ER + high, | 3.4 × 10⁴ | 122 | 1.92 | 2.4 |
| PR −, unknown ploidy, Ki-67 2% (low), | | | | |
| HER-2/neu 42% (high), p53 negative] | | | | |
| Tumor D (ILC) [ER + low, PR + high, | 3.0 × 10⁴ | 130 | 2.35 | 4.4 |
| diploid, S-phase 7.2% (high)] | | | | |
| Nonmalignant | | | | |
| Human breast cells | | | | |
| MCF10A | 4.0 × 10⁴ | 66 | 1.18 | 1 |
| Hs578Bst | 4.0 × 10⁴ | 113 | 1.50 | 1 |
| Tissue A | 1.0 × 10⁴ | 13 | 0.70 | 1 |
| Tissue B | 1.0 × 10⁴ | 18 | 0.96 | 1 |
| Tissue C | 1.0 × 10⁴ | 15 | 0.80 | 1 |
| Tissue D | 1.0 × 10⁴ | 10 | 0.54 | 1 |
| Benign breast pathology | | | | |
| Juvenile fibroadenoma | 4.0 × 10⁴ | 13 | 0.17 | N/A |
| Fibroadenoma | 4.0 × 10⁴ | 22 | 0.30 | N/A |
| Benign phyllodes tumor | 4.0 × 10⁴ | 33 | 0.44 | N/A |
| Ductal epithelial hyperplasia without alypia | 4.6 × 10⁴ | 17 | 0.35 | N/A |
| Normal breast cells^{f} | | | | N/A |
| Normal 1 | 1.0 × 10⁴ | 12 | 0.64 | N/A |
| Normal 2 | 2.0 × 10⁴ | 22 | 0.59 | N/A |
| Primary breast cell culture 1 | 4.0 × 10⁴ | 65 | 0.87 | N/A |
| ^{a}Values represent the relative number of errors created per nucleotide of the replicated plasmid. This derivation was based on the following formula described by Roberts and Kunkel (17): no. of mutant colonies/total no. of transformed colonies − background mutation frequency (no mutations detected in 5 × 10⁻⁸ colonies)/chance of a nucleotide defect within the lacZα gene if the colony | | | | |
| # expresses a white phenotype (0.5)/no. of sites in the target gene (373 bp). The lacZα gene comprises 8.25% of the total pBK-CMV plasmid (4518 bp). Each value reported in the table represents the average of at least three individual experiments, and the values did not deviate from the average by more than 5%. | | | | |

^{b}Values represent the fold increase in mutation frequency of the malignant cell synthesome, as compared to its genetically matched nonmalignant cell counterpart.
^{c}Although it is not a genetically matched cell line, the fold mutation for the MCF7 cell-derived synthesome was calculated using the mutation frequency measured for MCF10A cells. All other fold mutation calculations were made between genetically matched cell lines; N/A, no genetically matched counterpart available.
^{d}Surgically resected female human breast tissue. Genetically matched samples are denoted by corresponding alphanumeric designations (tumor A, tissue A, and so on). Factors such as stage of malignancy, genetics, race, and age were double blind during data collection.
^{e}IDC, infiltrating ductal carcinoma; ILC, infiltrating lobular carcinoma, determined by pathological diagnosis of tumor tissue.
^{f}Surgically resected breast reduction tissue from healthy females used to derived synthesome from frozen sample (tissue A) or from primary cultures (primary culture sample).

TABLE 2
DNA Replication Activity of the Genetically Matched
Malignant and Nonmalignant Breast DNA Synthesome

| | Units of T-antigen dependent | Fold T antigen dependent |
|---|---|---|
| Source of DNA Synthesome | DNA replication activity (×10⁻²)^{a} | replication activity^{b} |
| Malignant human breast cells | | |
| Tumor A^{c} (IDC^{d}) | 11.5 | 0.8 |
| Tumor B (IDC) | 11 | 1.3 |
| Tumor C | 23.5 | 2.0 |
| Tumor D | 12.5 | 1.0 |
| Average | 14.6 | 1.3 |
| Non malignant human breast cells | | |
| Tissue A | 15.0 | 1.0 |
| Tissue B | 8.7 | 1.0 |
| Tissue C | 11.5 | 1.0 |
| Tissue D | 11.5 | 1.0 |
| Average | 11.7 | 1.0 |

^{a}The values represent an average of two independent experiments. Replication values deviated by less then 3% from the average.
^{b}Fold DNA replication was calculated by dividing the units of replication observed for the malignant breast cell DNA synthesome by the replication units observed for the DNA synthesome isolated from the genetically matched nonmalignant breast cells. Each value represent the average of at least two independent experiments. Replication values deviated by less then 3% from the average.
^{c}Surgically resected female human breast tissue. Genetically matched samples are denoted by the corresponding letter (tumor A = tissue A). Factors such as stage of malignancy, genetics, race, and age were double blinded during data collection
^{d}IDC, infiltrating ductal carcinoma; ILC, infiltrating lobular carcinoma, determined by pathological diagnosis of tumor tissue.

### Example 2

### Altered PCNA in Malignant Breast Cells and Tissues

This example demonstrates that the DNA synthesome component PCNA is structurally altered in malignant breast cells and tissues.

### Example 2A

### Human Breast Cells

To determine whether PCNA is structurally altered in malignant breast cells, DNA synthesomes were isolated from four established human breast cell lines, MDA-MB468, Hs578T, MCF-10A and MCF-7, using our published procedures (Coll et al., Oncol. Res. 8: 435, 1996). Non-malignant primary breast cells were prepared from a human breast reduction sample as described by Stampfer (Stampfer, Tissue Culture Methods, 9:107-115, 1985). The malignant breast cell lines MCF-7, MDA-MB-468, and Hs578T, produce tumors in animal breast cancer models (H. D. Soule et al, J. Natl. Cancer Inst. 5: 1409 (1973), while the nonmalignant breast cell line MCF-10A does not (Soule, et al, Cancer Res. 50: 6075 (1990), Tait, et al Cancer Res. 5: 6087 (1990)).

Thirty micrograms of the DNA synthesome protein isolated from each of the five cells/cell lines (MDA-MB468, Hs578T, MCF-7, MCF-10A and primary breast cells) were subjected to individual two-dimensional polyacrylamide gel electrophoresis (2D-PAGE). The polyacrylamide gel was comprised of 9.2 M urea, 4% acrylamide, 2% ampholytes, and 20% Triton X-100. Polypeptides were separated along a pH gradient created using 100 mM NaOH and 10 mM H₃PO₄. The tube gels were then placed onto an 8% acrylamide SDS gel, and the polypeptides were separated by molecular weight.

The gels containing the resolved synthesome polypeptides were transferred at 20 volts for 18 hours to nitrocellulose filters. Western blot analyses of the filters were then performed using an antibody directed against the 36 kD PCNA polypeptide (PC 10, Oncogene Science) at a dilution of 1:1000. The PCNA profile in the Western blots was revealed by a light-enhanced chemiluminescence method (Amersham ECL).

A comparison of the mobility and abundance of the PCNA component of the cell derived DNA synthesome indicates a clear and significant difference in this protein's 2D-PAGE profile for the malignant versus non-malignant cell types. The results are shown in FIGS. 5A-to 5E. Specifically, malignant cells MCF-7 (FIG. 5A), Hs578T (FIG. 5C), and MDA-NIB-468 (FIG. 5E) all contain the acidic form of PCNA that is unique to malignant cells. Non-malignant cells MCF-10A (FIG. 5B) and nonmalignant primary breast cells (FIG. 5D) only contain the basic form of PCNA.

The PCNA associated with the nonmalignant MCF-10A synthesome is a single species and exhibits a basic pI (FIG. 5D), as is the PCNA from primary breast epithelial cells (FIG. 5D). The malignant cell synthesome displays two species of PCNA (FIGS. 5A, C, and E), a less abundant species and a more abundant species. The less abundant PCNA species has a mobility and basic pi that correspond exactly with those observed for the nonmalignant synthesome. The more abundant PCNA species of the malignant cell-derived synthesome has an acidic pI.

Thus, malignant cell DNA synthesomes contain a species of PCNA which is altered when compared to the PCNA of nonmalignant DNA synthesomes.

### Example 2B

### Human and Mouse Breast Tumors and Breast Tissues

This example demonstrates that breast tumors also express the acidic form of PCNA. The DNA synthesome was isolated from a virally induced mouse breast tumor and from six human lobular breast cancer tissues and four ductal breast cancer tissues. For comparison, PCNA associated with DNA synthesomes from nonmalignant breast tissue from two sources were analyzed: tissue excised during breast reduction surgery and nonmalignant tissue taken from patients with breast tumors.

Proteins of the DNA synthesome isolated from these tissues were resolved by 2D PAGE, transferred to nitrocellulose membranes, and probed with an antibody directed against PCNA, as described above.

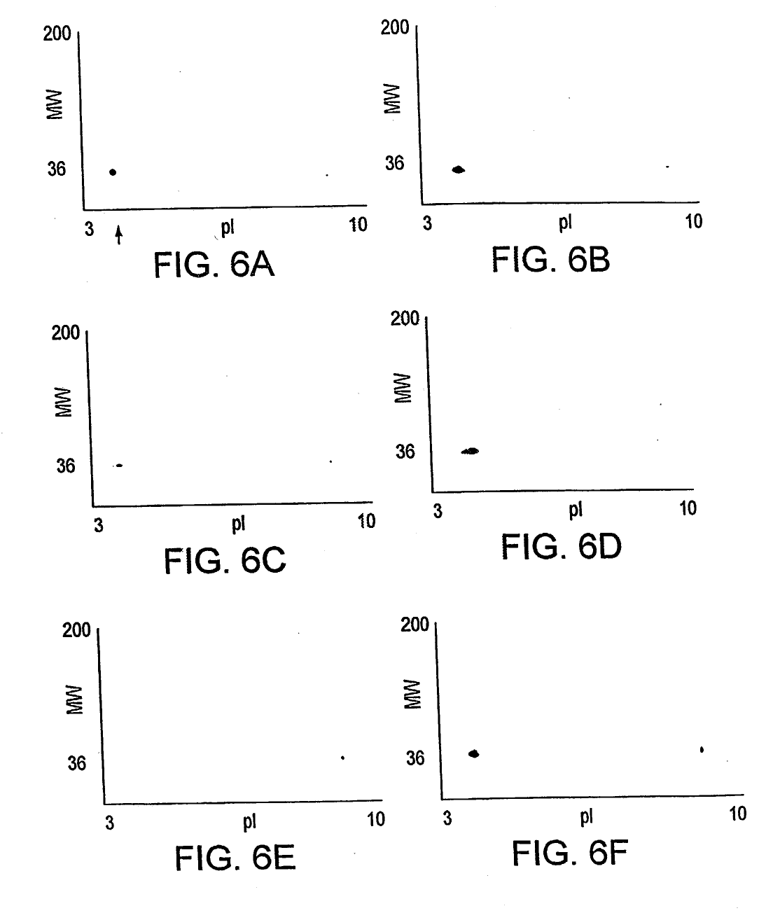

It was observed that PCNA derived from both mouse and human tumor tissue had a 2D PAGE profile consistent with that of the malignant breast cell lines, exhibiting both an acidic and a basic form of PCNA. In the Western blots of nonmalignant breast synthesome proteins, either the basic form of PCNA or no PCNA was detected (FIG. 6). Specifically, FIGS. 6A and 6B depict protein migration of PCNA from human ductal tumor; both acidic and basic forms of PCNA are present as is consistent with malignancy. FIGS. 6C and 6D depict protein migration of PCNA from human lobular tumor; both acidic and basic forms of PCNA are present as is consistent with malignancy. FIG. 6E depicts protein migration of PCNA from non-malignant human breast tissue; only the basic form of PCNA is present as is consistent with healthy, disease-free tissue. FIG. 6F depicts protein migration of PCNA from mouse breast tumor; both acidic and basic forms of PCNA are present as is consistent with malignancy. The tissues assayed in FIGS. 6C and 6E are genetically matched (taken from the same patient). The nonmalignant tissue sample is derived from the nonmalignant tissues adjacent to the malignant tissues sampled.

Thus, malignant breast tissue expresses the altered (acidic) form of PCNA expressed in malignant breast cell lines.

### Example 2C

### Cancer Specific PCNA In Transformed Cell Lines

This example demonstrates that the appearance of the acidic form of PCNA is specifically associated with the malignant transformation of a cell.

The cell line A1N4, a nonmalignant immortalized breast epithelial cell line, was transformed by the oncogenes c-myc and SV40T antigen to establish two stable cell lines: A1N4myc and A1N4T, respectively. The A1N4, A1N4myc, and A1N4T cell lines are not tumorigenic in nude mice. The DNA synthesome was isolated from the nonmalignant breast cell line A1N4 and the transformed, nonmalignant breast cell lines A1N4myc and A1N4T by the procedure described in detail above. The components separated by 2D PAGE as described in previous examples. Western blot analysis using an antibody directed against PCNA are shown for each cell line in FIG. 7. The parental cell line, A1N4 (FIG. 7A), does not contain the cancer specific, acidic form of PCNA. However, the non-malignant cell lines transformed with the c-myc gene and the SV40 T antigen, A1N4myc (FIG. 7B) and A1N4T (FIG. 7C), do contain the altered form of PCNA specifically associated with cancer. Overexpression of the c-myc gene in the A1N4 cell line with SV40T-antigen resulted in the overexpression of only the acidic form of PCNA.

Thus, these findings demonstrate that the altered form of PCNA results from oncogenic cell transformation events.

### Example 3

### The Malignant Form of PCNA in Other Cells and Tissues

This example demonstrates that the altered PCNA is found in other cancer types.

The DNA synthesome was isolated from LNCaP, PC50, KGE90, KYE350, HL60, HeLa, T98 and leukemia cell pellets according to published procedures (Coll et al., 1996) with minor modifications. Briefly, cell pellets were Dounce homogenized in a buffer containing 200 mM sucrose, 50 mM Hepes, 5 mM KCl, 2 mM DTT and 0.1 mM PMSF and centrifuged at 2500 rpm for 10 min to remove cell debris and pellet the nuclei. EDTA and EGTA were added to a final concentration of 5 mM to the supernatant. One volume of nuclear extraction buffer (350 mM KCl, 50 mM Hepes, 5 mM MgCl₂, 5 mM EDTA, 5 nM EGTA, 1 mM DTT, 0.1 mM PMSF) was added to the pellet and rocked at 4° C. for 2 hr. The supernatant was centrifuged at 18,000 rpm for 3 min. The supernatant was removed and centrifuged at 60,000 rpm for 22 min. The resulting supernatant was collected (S3 fraction). The nuclear pellet was centrifuged at 22,000 rpm for 3 min. The supernatant was removed and centrifuged at 60,000 rpm for 15 min. The resulting supernatant (NE fraction) was combined with the S3 fraction, layered on top of a 2 M sucrose cushion and centrifuged overnight at 40,000 rpm. The synthesome fraction was collected for analysis.

### Example 3A

### Prostate Cancer Cells

The DNA synthesomes were isolated from prostate cancer cells using our published procedures (Coll et al., Oncol. Res. 8: 435, 1996). The malignant cell lines, LnCAP and PC 10, produce tumors in animal prostate cancer models.

Thirty micrograms of the DNA synthesome protein isolated from each of the malignant cell lines, LnCAP and PC 10, were subjected to individual two-dimensional polyacrylamide gel electrophoresis (2D-PAGE). The polyacrylamide gel was comprised of 9.2 M urea, 4% acrylamide, 2% ampholytes, and 20% Triton X-100. Polypeptides were separated along a pH gradient created using 100 mM NaOH and 10 mM H₃PO₄. The tube gels were then placed onto an 8% acrylamide SDS gel, and the polypeptides were separated by molecular weight. To compare directly the PCNA species in the two cell types, a third 2D-PAGE was performed on a mixture of 30 micrograms each of the LnCAP and PC 10 synthesome proteins.

The gels containing the resolved synthesome polypeptides were transferred at 20 volts for 18 hours to nitrocellulose filters. Western blot analyses of the filters were then performed using an antibody directed against the 36 kD PCNA polypeptide (PC 10, Oncogene Science) at a dilution of 1:1000. The PCNA profile in the three Western blots was revealed by a light-enhanced chemiluminescence method (Amersham ECL). The results are shown in FIGS. 8A and 8B. Specifically, the malignant cells from LnCAP (FIG. 8A) and PC 10 (FIG. 8B) contain the acidic form of PCNA that is unique to malignant cells.

Thus, malignant prostate cell DNA synthesomes contain a species of PCNA which is altered when compared to the PCNA of nonmalignant DNA synthesomes.

### Example 3B

### Malignant Esophageal-Colon Cancer Cells

The DNA synthesomes were isolated from esophageal-colon cancer cells using our published procedures (Coll et al., Oncol. Res. 8: 435, 1996). The malignant cell lines, KGE 90, KYE 350 and SW 48, produce tumors in animal esophageal-colon cancer models.

Thirty micrograms of the DNA synthesome protein isolated from each of the malignant cell lines, KGE 90, KYE 350 and SW 48, were subjected to individual two-dimensional polyacrylamide gel electrophoresis (2D-PAGE). The polyacrylamide gel was comprised of 9.2 M urea, 4% acrylamide, 2% ampholytes, and 20% Triton X-100. Polypeptides were separated along a pH gradient created using 100 μM NaOH and 10 mM H₃PO₄. The tube gels were then placed onto an 8% acrylamide SDS gel, and the polypeptides were separated by molecular weight. To compare directly the PCNA species in the two cell types, a third 2D-PAGE was performed on a mixture of 30 micrograms each of the KGE 90. KYE 350 and SW 48 synthesome proteins.

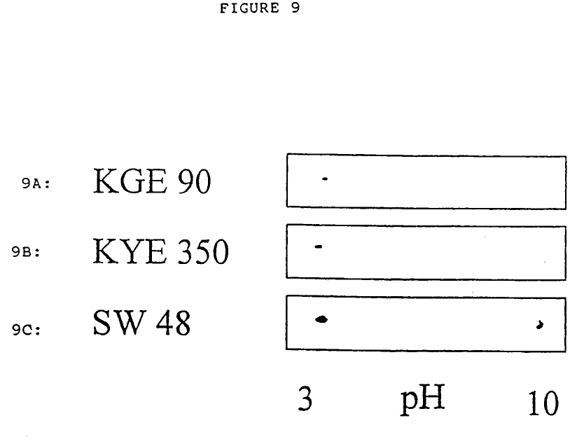

The gels containing the resolved synthesome polypeptides were transferred at 20 volts for 18 hours to nitrocellulose filters. Western blot analyses of the filters were then performed using an antibody directed against the 36 kD PCNA polypeptide (PC 10, Oncogene Science) at a dilution of 1:1000. The PCNA profile in the three Western blots was revealed by a light-enhanced chemiluminescence method (Amersham ECL). The results are shown in FIGS. 13A to 13C. Specifically, the malignant cells from KGE 90 (FIG. 9A), KYE 350 (FIG. 9B) and SW 48 (FIG. 9C) contain the acidic form of PCNA that is unique to malignant cells.

Thus, malignant esophageal-colon cell DNA synthesomes contain a species of PCNA which is altered when compared to the PCNA of nonmalignant DNA synthesomes.

### Example 3C

### Other Cancer Cells

The DNA synthesomes were isolated from esophageal-colon cancer cells using our published procedures (Coll et al., Oncol. Res. 8: 435, 1996). The malignant cell lines, HeLa (a cervical cancer cell line), T98 (a malignant glioma cell line) and H160 (a leukemia cell line), produce tumors in animal cancer models.

Thirty micrograms of the DNA synthesome protein isolated from each of the malignant cell lines, HeLa, T98 and H160, were subjected to individual two-dimensional polyacrylamide gel electrophoresis (2D-PAGE). The polyacrylamide gel was comprised of 9.2 M urea, 4% acrylamide, 2% ampholytes, and 20% Triton X-100. Polypeptides were separated along a pH gradient created using 100 mM NaOH and 10 mM H₃PO₄. The tube gels were then placed onto an 8% acrylamide SDS gel, and the polypeptides were separated by molecular weight. To compare directly the PCNA species in the two cell types, a third 2D-PAGE was performed on a mixture of 30 micrograms each of the HeLa. T98 and HL60 synthesome proteins.

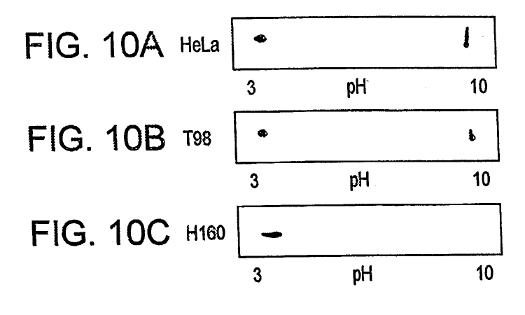

The gels containing the resolved synthesome polypeptides were transferred at 20 volts for 18 hours to nitrocellulose filters. Western blot analyses of the filters were then performed using an antibody directed against the 36 kD PCNA polypeptide (PC 10, Oncogene Science) at a dilution of 1:1000. The PCNA profile in the three Western blots was revealed by a light-enhanced chemiluminescence method (Amersham ECL). The results are shown in FIGS. 10A to 10C. Specifically, the malignant cells from HeLa (FIG. 10A). T98 (FIG. 10B) and HL60 (FIG. 10C) contain the acidic form of PCNA that is unique to malignant cells.

Thus, malignant cell DNA synthesomes contain a species of PCNA which is altered when compared to the PCNA of nonmalignant DNA synthesomes.

### Example 4

### The Malignant Form of PCNA

### Example 4A

### Ribosylatation of Altered PCNA

This example demonstrates that the acidic form of PCNA in malignant breast cells is not poly(ADP)-ribosylated.

Malignant MCF-7 cells were labeled with [³²-P]-NAD⁺. Malignant (MCF-7) and non-malignant (MCF-10) breast cell pellets were homogenized using 30 strokes of a Dounce homogenizer. One hundred micrograms of each homogenate was incubated with PCNA antibody. The level of PCNA antibody was sufficient to completely immunodeplete the fractions for the protein.

Following immunoprecipitation of the PCNA from the synthesome fractions the immunoprecipitated PCNA species were resolved by 2D-PAGE, as described above. The resolved polypeptides were then transferred to nitrocellulose filter membranes. Western blot analyses of the resolved PCNA polypeptides were then performed using anti-poly (ADP)-ribose moiety antibody (gift from Marc Smulson), at a dilution of 1:500. The Amersham ECL method was used to detect the immunoreactive species.

The Western blot thus obtained demonstrates that the unique form of PCNA in malignant cells, which has an acidic pI value (see Example 1, above), is not poly (ADP)-ribosylated (FIG. 13). The basic form of PCNA present in both malignant and nonmalignant cells is poly (ADP)-ribosylated.

### Example 4B

### Cell Proliferation Studies

This example demonstrates that the acidic form of PCNA detected in malignant cells is not a result of cell proliferation.

To determine whether the abundant levels of the acidic form of PCNA was a property unique to malignant breast cells as opposed to a proliferation response, the PCNA profile of PCNA isolated from estrogen-stimulated MCF-7 cells, control MCF-7 cells, and from benign proliferative breast tumors were analyzed. Estrogen has been shown to have a stimulatory effect on cellular proliferation (Levenson, A. & Jordan, V., Cancer Res. 57: 3071 (1997)).

Thirty to sixty micrograms of DNA synthesome were isolated from each cell or tissue by the processes described above.

It was found that the estrogen-stimulated cells had an increased rate of proliferation compared to control cells, as demonstrated by several parameters (³H Thymidine uptake, polymerase activity and flow cytometry) and as described by others (Levenson. A. & Jordan, V., Cancer Res. 57: 3071 (1997); Kyung-Sun, K. et al, Carcinogenesis 18: 251-77 (1997); M. Brown, Hematology/Oncology Clin. North Am. 8: 101 (1994)). See Table 3 below.

TABLE 3
Stimulation of Cell Proliferation Following Treatment with 17-β-estradiol

| | | | 17-β-Estradiol (E₂) | |
|---|---|---|---|---|
| Parameter | Control Cells^{a} | | treated cells^{b} | |
| [³H]-Thymidme uptake^{c} | 1,548 | cpm/10⁵ cells | 10,564 | cpm/10⁵ cells |
| DNA polymerase activity^{d} | 496 +/− 80 | cpm/mg | 1,359 +/− 118 | cpm/mg |
| Cells in S phase^{e} | 10.7% | | 60.1% | |

^{a}Control cells are MCF-7 cells that were grown in phenol red-free DMEM, which was supplemented with 10% dextran coated, charcoal treated fetal bovine serum, 1% penicillin/streptomycin, and non-essential amino acids.
^{b}17-β-estradiol (E₂) treated cells were grown for 48 hours under essentially the same conditions as the control along with the addition of 1 mM 17-β-estradiol to the medium.
^{c}[³H]-Thymidine uptake, according to the procedure described by Malkas et al., 1990.
^{d}DNA polymerase a activity was measured as described by Malkas et al., 1990.
^{e}Cell cycle distribution analyses of the cultured cells grown in the presence or absence of 17-β-estradiol were performed as described by Lin et al. (Lin et al., Cell Growth Differ., 8: 1359-1369, 1997)

The DNA synthesome from these cells was isolated and the components were resolved by 2D-PAGE and transferred to nitrocellulose membranes. The membranes were probed with anti-PCNA antibody, as described above. It was observed that the same 2D PAGE profile for both the control and estrogen-treated cells (see FIG. 11). Specifically, the 2D PAGE profile was found for estrogen treated MCF-7 cells (FIG. 11A) is identical to that of the untreated MCF-7 cells (FIG. 11B), both containing the acidic form of PCNA that is unique to malignancy. Benign breast tumor tissue (FIG. 11C) contained only the basic form of PCNA. Thus, acidic PCNA is a biomarker for malignancy, being absent from normal tissues or benign tumors.

DNA synthesomes from four benign proliferative breast tumors were also isolated. The 2D PAGE profile of synthesome proteins from these tumors displayed negligible levels of PCNA.

These results demonstrate that the acidic form of PCNA is unique to malignant cells and is not a result of cell proliferation.

These data provide compelling evidence that the acidic form of PCNA is characteristic of malignant breast cells.

### Example 4C

### Genetic Analysis of Altered PCNA

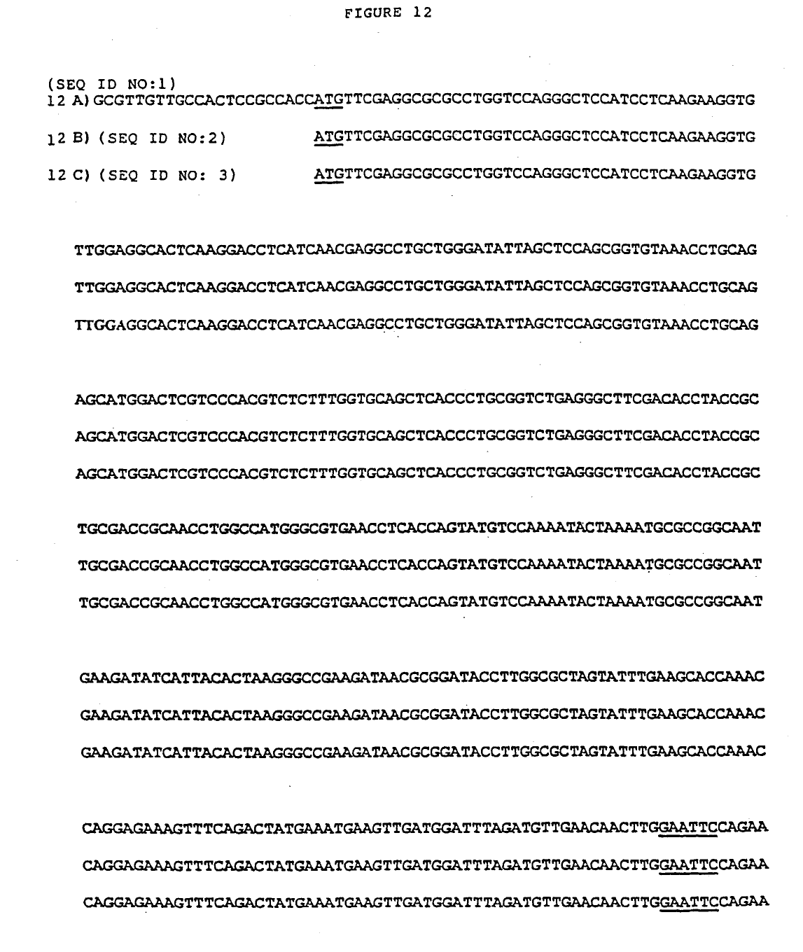

This example demonstrates that genetic mutation is not responsible for the acidic form of PCNA in malignant cells. Total cellular RNA isolated from MCF-7 and MCF-10A cells was used to clones the cDNA encoding the entire PCNA translation unit from each cell line. The cDNA was cloned from total cellular RNA isolated from exponentially growing MCF-7 and MCF-10A cells using reverse transcriptase PCT and the pCR2.1 vector. Four independent clones encoding the PCNA gene derived from MCF-7 cells and four independent clones from MCF-10A were sequenced. Ampicillin-resistant colonies containing the cDNA were chosen using the blue/white selection assay and Miniprep DNA was isolated from the selected colonies and given to the University of Maryland, Baltimore. Biopolymer Core Facility for nucleotide sequence analysis. Sequence analysis indicated that these eight independent clones have an identical nucleotide sequence (see FIG. 12, parts A-C, SEQ ID NOS.1-3). Furthermore, this nucleotide sequence does not differ from that of the published sequence for the PCNA gene clones from human lymphoma cell line MOLT-4 (FIG. 12A, Almendral et al, Proc. Natl. Acad. Sci. USA, 84: 1575-1579, 1987).

### Example 5

### The Malignant Form of PCNA in Body Fluid

This example demonstrates that the altered acidic form of PCNA can be readily detected in both blood and serum of cancer patients while not detected in the serum of cancer free patients. Specifically, the serum of a stage III breast cancer patient and the blood of chronic myelogenous leukemia (CML) and acute myelogenous leukemia (AML) patients were studied.

Regarding the leukemia samples, the CML samples were obtained from Dr. Moshe Talpaz through a collaboration with the MD Anderson Cancer Center. The AML sample was obtained from Dr. Lynn Abruzzo through a collaboration with the Greenebaum Cancer Center.

Serum collected from a patient with intraductal breast carcinoma was Dounce homogenized and centrifuged at 2500 rpm for 10 min. One volume of nuclear extraction buffer (350 mM KCl, 50 mM Hepes, 5 mM MgCl2, 5 mM EDTA, 5 mM EGTA, 1 mM DTT, and 0.1 mM PMSF) was added to the pellet and rocked at 4° C. for 2 hr. The nuclear pellet was centrifuged at 22,000 rpm for 3 min. The supernatant was collected and centrifuged at 60,000 rpm for 15 min. The supernatant was collected and used for analysis. The DNA synthesome was isolated and purified from the samples by the process described in detail above. The components of the synthesome were resolved by 2D PAGE and subjected to Western blot analyses using an antibody directed against PCNA. The electrophoretic mobility of PCNA was then measured.

Specifically, the DNA synthesome protein (20-40 mg) was loaded onto the first dimension tube gel (9.2 M urea, 4% acrylamide, 2% ampholytes (pH 3-10), and 20% Triton X-100). The polypeptides were separated along a pH gradient established using 100 mm NaOH and 10 mM H₃PO₄. The tube gels were placed onto an 8% acrylamide SDS gel, and the polypeptides were resolved by molecular weight. The proteins were then transferred electrophoretically to nitrocellulose membranes. An antibody directed against PCNA (PC 10. Oncogene Science) was used at a dilution of 1: 1000. Immunodetection of PCNA was performed using a light enhanced chemiluminescence system (Amersham).

### Example 5A

### PCNA in Breast Cancer Patients

To be potentially beneficial as a tumor marker for the detection of breast malignancy, the altered form of PCNA should be readily detectable in the serum of a patient with breast cancer. In evaluating cancer development, the analysis of serum samples for specific tumor markers has failed to identify satisfactory markers to use for diagnosis and for monitoring the progress of patients with breast cancer (Hayes, 1996; Schwartz et al., 1993). In general PCNA has not been very useful as a tumor marker for the prediction of patient outcome (Haerslev et al., 1996; Schmitt et al., 1994). The present study demonstrated that the altered form of PCNA can be readily detected in the serum collected from a patient with stage III intraductal breast cancer, and that PCNA is not detectable in the serum from cancer free individuals. This finding suggests that serum testing for PCNA may be beneficial for the detection of residual disease or disease recurrence in breast cancer patients.

To determine whether the malignant form of PCNA could be a useful marker for identifying individuals with breast cancer, serum collected from a breast cancer patient was examined for the presence of the acidic form of PCNA. A serum sample collected from a breast cancer patient with stage III intraductal breast carcinoma was analyzed by 2D PAGE and Western blot analysis using an antibody directed against PCNA. The Western blot analysis showed that the serum sample contained the altered form of PCNA (FIG. 14A). PCNA was not detected in control serum samples collected from two cancer free individuals. This result indicated that the cancer specific form of PCNA had been released into the peripheral blood from the tumor cells. Furthermore, the data indicate that nonmalignant cells do not release detectable levels of PCNA.

### Example 5

### PCNA in Leukemia Patients

The role of PCNA in the development and progression of CML is not well characterized. CML is a biphasic disease characterized by an early chronic phase followed by a blast phase (Zaccaria et al., 1995). Takasaki et al. (1984b) demonstrated a correlation between the number of leukocytes expressing PCNA and the percent of blast cells in blood during the blast phase of CML. These investigators also identified the presence of non-blast cells which were positive for PCNA in the peripheral blood during the blast phase of CML. This result differs from the observation that the non-blast cells were negative for PCNA in chronic phase (Takasaki et al., 1984). The PCNA labeling index for CML cells is not significantly different form normal bone marrow cells (Thiele et al., 1993). However, in the chronic myeloid proliferative disorder oslemyelofibrosis, there is a significant increase in the PCNA labeling index (Thiele et al., 1994). Interferon treatment resulted in decreased PCNA labeling. In the present study, the results demonstrated that the leukemia samples examined contain the altered form of PCNA, while samples collected from cancer free individuals did not contain the altered form of PCNA.

Blood samples from patients with chronic myelogenous leukemia (CML) and acute myelogenous leukemia (AML) were analyzed to determine whether the acidic form of PCNA was present. The protein migration patterns exhibited by PCNA are shown in FIG. 14. The acidic form of PCNA was identified in the AML sample (FIG. 14B) and all of the CML samples (FIGS. 14C-E).

### Example 5C

### PCNA Absent from Cancer Free Serum

Blood samples from cancer free patients were analyzed to determine whether the acidic form of PCNA was present. The DNA synthesome was isolated from the samples by the process described above. The components were resolved by 2D PAGE followed by Western blot analysis using an antibody directed against PCNA. The results of the migration pattern assay are shown in FIG. 15. The acidic form of PCNA was not detectable in the cancer free serum.

Although serum and blood are discussed in detail, it is clear that other readily available, easily obtainable body fluids, such as lymph, pleural fluid, spinal fluid, saliva, sputum, urine, and semen, can be utilized.

### Example 6

### Altered Components of DNA Synthesome in Malignant Cells

This example demonstrates that other components of the DNA synthesome are structurally altered in malignant cells.

### Example 6A

### Altered Form of Polymerase α

To determine whether Pol A (polymerase α) is structurally altered in malignant breast cells, DNA synthesomes were isolated from four established human breast cell lines, MCF-10A and MCF-7, using our published procedures (Coll et al., Oncol. Res. 8: 435, 1996). The malignant breast cell line, MCF-7, produces tumors in animal breast cancer models (H. D. Soule et al, J. Natl. Cancer Inst. 5: 1409 (1973), while the nonmalignant breast cell line MCF-10A does not (Soule, et al, Cancer Res. 50: 6075 (1990), Tait, et al Cancer Res. 5: 6087 (1990)).

Thirty micrograms of the DNA synthesome protein isolated from each of the cell lines (MCF-7 and MCF-10A) were subjected to individual two-dimensional polyacrylamide gel electrophoresis (2D-PAGE). The polyacrylamide gel was comprised of 9.2 M urea, 4% acrylamide, 2% ampholytes, and 20% Triton X-100. Polypeptides were separated along a pH gradient created using 100 mM NaOH and 10 mM H₃PO₄. The tube gels were then placed onto an 8% acrylamide SDS gel, and the polypeptides were separated by molecular weight.

The gels containing the resolved synthesome polypeptides were transferred at 20 volts for 18 hours to nitrocellulose filters. Western blot analyses of the filters were then performed using an antibody directed against the 120 kD Pol A polypeptide at a dilution of 1:1000. The Pol A profile in the Western blots was revealed by a light-enhanced chemiluminescence method (Amersham ECL).

A comparison of the mobility of the Pol A component of the cell derived DNA synthesome indicates a clear and significant difference in this protein's 2D-PAGE profile for the malignant versus non-malignant cell types. The results are shown in FIGS. 16A-and 16B. Specifically, malignant cells MCF-7 (FIG. 16A) contains two species of Pol A which differ in charge, one basic and one acidic. Non-malignant cells MCF-10A (FIG. 16B) only contain the single basic species of Pol A.

Thus, malignant cell DNA synthesomes contain a species of Pol A which is altered when compared to the Pol A of nonmalignant DNA synthesomes.

### Example 6B

### Altered Form of RP-A

To determine whether RP-A (replication protein A) is structurally altered in malignant breast cells, DNA synthesomes were isolated from four established human breast cell lines, MCF-10A and MCF-7, using our published procedures (Coll et al., Oncol. Res. 8: 435, 1996). The malignant breast cell line, MCF-7, produces tumors in animal breast cancer models (H. D. Soule et al, J. Natl. Cancer Inst. 5: 1409 (1973), while the nonmalignant breast cell line MCF-10A does not (Soule, et al, Cancer Res. 50: 6075 (1990), Tait, et al Cancer Res. 5: 6087 (1990)).

Thirty micrograms of the DNA synthesome protein isolated from each of the cell lines (MCF-7 and MCF-10A) were subjected to individual two-dimensional polyacrylamide gel electrophoresis (2D-PAGE). The polyacrylamide gel was comprised of 9.2 M urea, 4% acrylamide, 2% ampholytes, and 20% Triton X-100. Polypeptides were separated along a pH gradient created using 100 mM NaOH and 10 mM H₃PO₄. The tube gels were then placed onto an 8% acrylamide SDS gel, and the polypeptides were separated by molecular weight.

The gels containing the resolved synthesome polypeptides were transferred at 20 volts for 18 hours to nitrocellulose filters. Western blot analyses of the filters were then performed using an antibody directed against the RP-A polypeptide at a dilution of 1:000, The RPA profile in the Western blots was revealed by a light-enhanced chemiluminescence method (Amersham ECL).

A comparison of the mobility and abundance of the RP-A component of the cell derived DNA synthesome indicates a clear and significant difference in this protein's 2D-PAGE profile for the malignant versus non-malignant cell types. The results are shown in FIGS. 17A and 17B. Specifically, malignant cells MCF-7 (FIG. 17A) contain two species of the 70 kDa subunit whereas non-malignant cells, MCF-10 (FIG. 17B) contain only a single species. The form of the RP-A that is unique to the malignant cells was found to be of a higher molecular weight than that found in the non-malignant cell species of RP-A and of more abundance. In contrast to the alterations associated with PCNA and Pol A, the cancer specific form of RP-A does not appear to change in charge.

Thus, malignant cell DNA synthesomes contain a species of RP-A which is altered when compared to the RP-A of nonmalignant DNA synthesomes.

### Example 7

### The Components of the DNA Synthesome

As discussed in detail above, the DNA synthesome is a multiprotein DNA replication complex which is present in mammalian cells. The complex of proteins is fully competent to replicate DNA in vitro (Applegren et al., 1995; Lin et al., 1996; Tom et al., 1996). Further to these findings, it was discovered by the inventors that the DNA synthetic and the DNA mismatch repair (MMR) proteins work together to mediate the high degree of fidelity exhibited during the cellular DNA replication process. Disclosed herein is evidence of the structural and functional interaction of the core components of the human DNA synthesome with the DNA MMR proteins, hMSH2, hMLH1, hMSH6, hPMS1, hPMS2, MYH, and Ku 80. Western blot and co-immunoprecipitation analyses of HeLa cell sucrose gradient fractions containing the peak replication activity mediated by the highly purified DNA synthesome indicate that these MMR proteins are tightly associated with the core components of the purified synthesome. In addition, the purified DNA synthesome demonstrates both a high level of DNA replication activity and an exquisite binding specificity for templates containing heterogenous single base mispairs and insertion-deletion loops of two to four nucleotides. Recognition of these types of mismatch by the DNA synthesome further indicate the premise that the mismatch repair proteins are tightly associated with the DNA synthesome, the MMR proteins retaining function throughout the purification of the synthesome. The results described herein demonstrate that the MMR proteins are components of the DNA synthesome and that recognition of replicative errors must occur shortly after the daughter DNA strands are synthesized.

### Example 7A

### Protein-Protein Interactions

To demonstrate the strength of the bond between replication and repair components of the DNA synthesome, we performed co-purification and co-precipitation studies directed to testing the protein-protein interactions of the components of the synthesome.

Suspension cultures of HeLa cells were grown and harvested according to published procedures. The DNA synthesome was isolated from the HeLa cells and the replication and polymerase activities determined by the processes described above. The results showed that DNA polymerase alpha (Pol A), DNA polymerase delta (Pot D) and the SV40 in vitro DNA replication activities were found exclusively in the sucrose gradient fractions 4-7, with the peak activities concentrated in fraction 5.

Using 2D-PAGE analyses, 30 to 100 micrograms of sucrose gradient fractions per lane were resolved and electophoretically transferred to nitrocellulose membranes. Immunodetection of specific DNA mismatch repair and replication proteins were carried out using a light enhanced chemiluminescent (ECL) detection system as discussed above. The individual antibodies directed against MMR proteins, hMSH2 hMLH1, hMSH6, hPMS2, and hPMS1 were used at a dilution of 1 microgram/milliliter (Santa Cruz Biotechnology, Santa Cruz, Calif.). The antibody directed against MYH was used at a dilution of 1:200 (a gift from Dr. Anindya Dutta). The antibody directed against Ku-80 (Sigma) was used at a dilution of 1:1000. The antibody directed against PCNA (Oncogene Science) was used at a dilution of 1:1000. The antibody directed against Pol A (SJK 132-20) was used at a dilution of 1:500. The appropriate species-specific horseradish peroxidase conjugated secondary antibodies were used to visualize the position of these specific replication and repair proteins on the immunoblots. Prestained SDS-PAGE molecular size markers were obtained from New England Biolabs (Boston, Mass.). The results are shown in FIG. 18.

Co-immunoprecipitation reactions were carried out using 100 micrograms of the sucrose gradient fraction (fraction 5) with antibody directed against DNA polymerase delta, PCNA, and hMSH2 according to the modified procedure in Coll et al. (1997). Summary of the data is provided in Table 4 below.

Proteins Co-Immunoprecipitated

| Precipitating | DNA | DNA | | | | | | | | |
|---|---|---|---|---|---|---|---|---|---|---|
| Antibody | Pol A | Pol D | PCNA | hMSH2 | hMSH6 | hMLH1 | hPMS1 | hPMS2 | Ku80 | MYH |
| DNA Pol D | + | + | + | − | + | + | + | + | + | + |
| PCNA | + | + | + | + | + | + | + | + | + | + |
| hMSH2 | + | − | + | + | + | + | +L | +L | − | + |

+L = as suggested by Acharya et al., 1996 and Unar et al., 1996

### Example 7B

### Repair Specifically Associated with Replication

Oligonucleotides of 40 base pairs containing a single G/T, A/G, or an A/7,8-dihydro-8-oxodeoxyguanine (A/GO) mispair, or a single insertion-deletion loop of 2 or 4 nucleotides were constructed by the University of Maryland Core Biopolymer Facility (UMB). After the oligomers were annealed to create the heteroduplex and homoduplex templates, they were 3′ end labeled with the Klenow fragment of E. coli DNA polymerase I for 30 minutes at 25° C. in the presence of an [alpha-³²-P] dCTP (50 microCi at 3,000 Ci/mmol), 20 micromolar dTTP, 20 micromolar dATP, 20 micromolar dTGP. The resulting blunt ended 40 bp duplex DNA mixture was passed through a 1 ml Biogel P-60 column to remove unincorporated nucleotides.

The binding reaction consisted of one microgram of sucrose gradient peak purified DNA synthesome which was incubated with 1.8 fmol of the labeled template for 20 minutes at 37° C., after which glutaraldehyde was added to a final concentration of 0.1% and the reaction incubated for an additional 10 minutes at 25° C. After sucrose was added to a final concentration of 14% in the reaction mixture, the bound protein-DNA complexes were resolved at 4° C. through a 5% non-denaturing polyacrylamide gel using 125V for 1 hour. The gels were then dried and exposed to Kodak XAR-5 film (Kodak, Inc Rochester, N.Y.) at −80° C. for 12-19 hours.

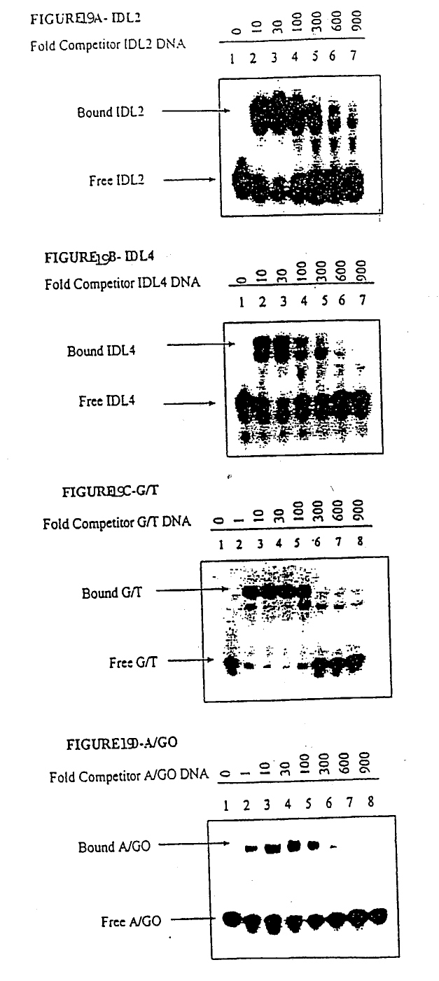

Heteropolymer competition reactions demonstrated (FIGS. 19A-19D) that the synthesome binding reaction with the DNA template containing a single nucleotide mismatch or IDL (comprised of 2 or 4 mismatched nucleotides) can be competed away completely by the corresponding unlabeled DNA template containing a single mismatch or IDL.

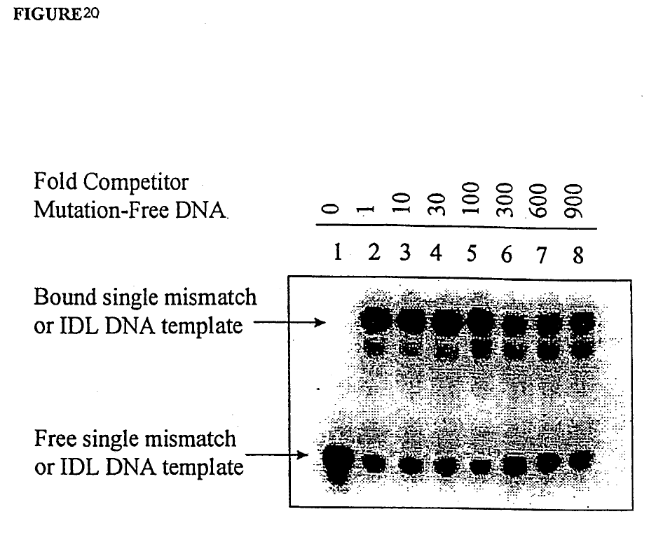

Homopolymer competition assay included unlabeled competitor homopolymer DNA (perfectly matched DNA containing no mismatch or IDL) in a range of concentrations in which the competitor was present at up to 900 fold above that of the labeled template. These assays demonstrated that the binding of the DNA synthesome to DNA templates containing a G/T, A/G, or A/GO mispair or and IDL2 or IDL4 could not be competed away by a homopolymer DNA template containing no mismatches. The results, shown in FIG. 20, indicate that the DNA synthesome has a higher affinity for DNA containing mismatches (regardless of type of mismatch) than a perfectly matched DNA template. FIG. 20 shows a typical result of the homopolymer competition assay, the assay using labeled heteroduplex template containing a GOT mismatch and unlabeled competitor (identical to the heteroduplex DNA sequence in all matched positions).

These findings support the new model of the DNA synthesome (shown in FIG. 1). The model includes the DNA MMR components as well as the DNA replication components.

In conclusion, one of the hallmarks of malignancy is the accumulation of genetic mutations which contribute to genetic instability exhibited by many types of cancer cells. Some of these mutations are postulated to contribute to the uncontrolled cellular proliferation observed for most tumors. The accumulation of genetic errors in cancer cells is relatively high, particularly considering the fact that nonmalignant cells are estimated to make an average of 1.4×10⁻¹⁰ mutations/base pair/cell division (Cheng and Loeb, 1993; Loeb 1998). Following the initial observation that the DNA replication apparatus of malignant and nonmalignant breast cells was itself mutagenic, the inventors herein have discovered that structural differences in specific DNA replication proteins exist between malignant and nonmalignant breast cells (see also Sekowski et al. 1998, specifically incorporated herein by reference in its entirety). Structurally altered forms of PCNA, RP-A and Polymerase α are discussed in detail. Furthermore, it is clear that various other components of the DNA synthesome are also altered in the malignant form (see FIG. 2). These altered forms can serve as biomarkers of malignancy and can be easily measured and quantified to diagnose and prognose many forms of cancer.

While the invention has been described in detail, and with reference to specific embodiments thereof, it will be apparent to one with ordinary skill in the art that various changes and modifications can be made therein without departing from the spirit and scope thereof. All references cited herein are incorporated by reference in their entirety.

What is claimed:

1. An isolated and purified preparation of antibodies which specifically bind a component of a DNA synthesome which is altered in a malignant cell.

2. The isolated and purified preparation of antibodies of claim 1 wherein the altered component of the DNA synthesome is selected from the group consisting of the altered species of proliferating cell nuclear antigen (PCNA), DNA polymerase alpha (Pol A), and replication protein A (RPA).

3. The isolated and purified preparation of antibodies of claim 1 wherein the antibodies are monoclonal.

4. The isolated and purified preparation of antibodies of claim 1 wherein the antibodies are polyclonal.

5. The isolated and purified preparation of antibodies of claim 1 wherein the antibodies are affinity purified.

6. The isolated and purified preparation of antibodies of claim 1 wherein the antibodies are obtained using a phage display method.

7. A method for diagnosing or prognosing malignancy comprising the step of detecting an alteration in a DNA synthesome of a body fluid sample obtained from a patient suspected of having a malignant condition, wherein the alteration in a component of the DNA synthesome indicates the presence of a malignant cell or component thereof in the fluid sample.

8. The method of claim 7 wherein the body fluid is selected from the group consisting of blood, plasma, lymph, serum, pleural fluid, spinal fluid, saliva, sputum, urine, and semen.

9 The method of claim 7 wherein the body fluid is a circulated fluid

10 The method of claim 7 wherein the body fluid is blood.

11. The method of claim 7 wherein the alteration in the DNA synthesome is an altered form of one of the group consisting of PCNA, Pol A, and RP-A

12. The method of claim 7 wherein the alteration in the DNA synthesome is an altered form of PCNA.

13. A method to aid in diagnosing or prognosing malignancy, comprising the step of detecting an alteration in a DNA synthesome of a tissue sample, wherein cells of the tissue sample are suspected of being malignant, wherein the alteration in the DNA synthesome indicates the presence of a malignant cell in the tissue sample.

14. The method of claim 13 wherein the alteration in the DNA synthesome is detected with an antibody which specifically binds to the altered component of the DNA synthesome.

15. The method of claim 13 wherein the alteration in the DNA synthesome is an altered form of at least one of the group consisting of proliferating cell nuclear antigen (PCNA), DNA polymerase alpha (Pol A) and replication protein A (RP-A).

16. The method of claim 12 wherein the alteration in the DNA synthesome is detected by
detecting a first level of DNA replication fidelity in a first tissue sample, wherein cells of the first tissue sample are nonmalignant,
detecting a second level of DNA replication fidelity in a second tissue sample, wherein the cells of the second tissue sample are suspected of being malignant; and
comparing the first and second levels of DNA replication fidelity, wherein a lower level of DNA replication fidelity in the second tissue sample indicates the presence of cells in the second tissue samples which are malignant.

17. The method of claim 12 wherein the tissue which is suspected of containing cells which are malignant is selected from the group consisting of breast, blood, prostate, brain, esophageal, cervical, and colon.

18. The method of claim 17 wherein the tissue is breast tissue.

19. A method of detecting the presence of metastatic malignancy, comprising the steps of:
contacting a blood sample of a patient suspected of having a metastatic neoplasm with an antibody which specifically binds to a component of a DNA synthesome which is altered in a malignant cell; and
observing a pattern of specific binding of the antibody to cells in the blood, wherein specific binding of the antibody to the cells in the blood sample indicates the presence of malignant cells or a component thereof in the blood sample.

20. A method of screening test compounds for the ability to suppress a malignant phenotype of a cell, comprising the steps of contacting a malignant cell with a test compound; and
detecting an altered component of a DNA synthesome in the malignant cell, a test compound which inhibits the function of the altered component of the DNA synthesome in the malignant cell being a potential therapeutic agent for treating malignancy.

21. The method of claim 20 wherein the altered component of the DNA synthesome is detected with an antibody which specifically binds to said component of the DNA synthesome.

22. The method of claim 21 wherein the antibody specifically binds to said altered component of the DNA synthesome is selected from the group consisting an altered species of proliferating cell nuclear antigen (PCNA), an altered species of DNA polymerase alpha (Pol A), and an altered species of replication protein A (RPA).

23. The method of claim 20 wherein the altered property of the DNA synthesome is a decreased level of DNA replication fidelity in the malignant cell.

24. A kit for diagnosing or prognosing malignancy, comprising an antibody which specifically binds to a component of a DNA synthesome which is altered in a malignant cell.

25. The kit of claim 24 wherein the antibody specifically binds to a component of a DNA synthesome is selected from the group consisting an altered species of proliferating cell nuclear antigen (PCNA), an altered species of DNA polymerase alpha (Pol A), and an altered species of replication protein A (RPA).

26 A kit for screening test compounds for the ability to suppress a malignant phenotype of a cell, comprising.
an isolated and purified antibody which specifically binds to a component of a DNA synthesome which is altered in the malignant cell; and
a sample of viable malignant cells.

27. The kit of claim 26 wherein the altered component of the DNA synthesome is selected from the group consisting an altered species of proliferating cell nuclear antigen (PCNA), an altered species of DNA polymerase alpha (Pol A), and an altered species of replication protein A(RPA).

28. A method of restoring normal function of a DNA synthesome in a malignant cell, comprising the step of contacting the cell with an antibody which specifically binds to a component of the DNA synthesome which is altered in the malignant cell, wherein the normal function of the DNA synthesome is restored.

29. The method of claim 28 wherein the altered component of the DNA synthesome is selected from the group consisting an altered species of proliferating cell nuclear antigen (PCNA), an altered species of DNA polymerase alpha (Pol A), and an altered species of replication protein A (RPA).

30. A therapeutic composition for restoring normal function of a DNA synthesome in a malignant cell, comprising:
an antibody which specifically binds to a component of the DNA synthesome which is altered in the cell; and
a pharmacologically suitable excipient.

31. The therapeutic composition of claim 30 wherein the altered component of the DNA synthesome is selected from the group consisting an altered species of proliferating cell nuclear antigen (PCNA), an altered species of DNA polymerase alpha (Pol A), and an altered species of replication protein A (RPA).

32 A method of detecting the presence of malignancy, comprising the steps of
contacting a blood sample of a patient suspected of having a metastatic neoplasm with an antibody which specifically binds to a component of a DNA synthesome which is altered in a malignant cell, and
observing a pattern of specific binding of the antibody to cells in the blood, wherein specific binding of the antibody to the cells in the blood sample indicates the presence of malignant cells or a component thereof in the blood sample.

33. A method of screening test compounds for the ability to suppress a malignant phenotype of a cell, comprising the steps of:
contacting a malignant cell with a test compound; and
detecting an altered component of a DNA synthesome in the malignant cell, a test compound which blocks the function of the altered component of the DNA synthesome in the malignant cell being a potential therapeutic agent for treating malignancy.

34. A method of blocking the abnormal function of a DNA synthesome in a malignant cell, rendering the malignancy static and halting the growth of the malignancy, comprising the step of contacting the cell with an antibody which specifically binds to a component of the DNA synthesome which is altered in the malignant cell, wherein the abnormal function of the DNA synthesome is blocked.

35. The method of claim 34 wherein the altered component of the DNA synthesome is selected from the group consisting an altered species of proliferating cell nuclear antigen (PCNA), an altered species of DNA polymerase alpha (Pol A), and an altered species of replication protein A (RPA).
